(19) United States
(12) Patent Application Publication
(10) Pub. No.: US 2007/0027130 A1
(43) Pub. Date: Feb. 1, 2007

(54) Tricyclic 6-alkylidene-penem beta-lactamase inhibitors and beta-lactam antibiotic combination: a broad spectrum antibiotic

(75) Inventors: Tarek S. Mansour, New City, NY (US); Aranapakam M. Venkatesan, Rego Park, NY (US); Patricia Bradford, Nyack, NY (US); Peter J. Petersen, Chester, NY (US); Steven J. Projan, Boston, MA (US)

Correspondence Address: WILMER CUTLER PICKERING HALE AND DORR LLP /, 60 STATE STREET, BOSTON, MA, 02109, US

(73) Assignee: Wyeth, Madison, NJ (US)

(21) Appl. No.: 11/493,331

(22) Filed: Jul. 26, 2006

Related U.S. Application Data
(60) Provisional application No. 60/702,808, filed on Jul. 27, 2005.

(51) Int. Cl. A61K 31/424; A61K 31/43

(52) U.S. Cl. 514/193; 514/210.06

(57) ABSTRACT

The present invention provides a β-lactam antibiotic such as cefepime and a compound of formula I, pharmaceutical compositions and the use thereof for the treatment of bacterial infection or disease in a patient in need thereof.

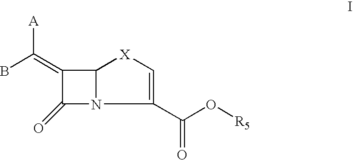

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF INVENTION

This invention relates to certain tricyclic 6-alkylidene penems which act as broad spectrum β-lactamase inhibitors, when combined with a β-lactam antibiotic, including a “fourth-generation” cephalosporin antibiotic such as cefepime, a penicillin antibiotic, or a carbapenem antibiotic. β-Lactamases hydrolyze β-lactam antibiotics, and as such serve as the primary cause of bacterial resistance. The compounds of the present invention when combined with a β-lactam antibiotic such as cefepime provide an effective treatment against life threatening bacterial infections.

### BACKGROUND OF THE INVENTION

Penicillins and cephalosporins are the most frequently and widely used β-lactam antibiotics in the clinic. However, the development of resistance to β-lactam antibiotics by different pathogens has had a damaging effect on maintaining the effective treatment of bacterial infections. (Coleman, K. Expert Opin. Invest. Drugs 1995, 4, 693; Sutherland, R. Infection 1995, 23 (4) 191; Bush, K, Cur. Pharm. Design 1999, 5, 839-845). The most significant known mechanism related to the development of bacterial resistance to the β-lactam antibiotics is the production of Class-A, Class-B and Class-C serine β-lactamases. These enzymes degrade the β-lactam antibiotics, resulting in the loss of antibacterial activity. Class-A enzymes preferentially hydrolyze penicillins whereas Class-C lactamases have a substrate profile favoring cephalosporin hydrolysis. (Bush, K.; Jacoby, G. A.; Medeiros, A. A. Antimicrob. Agents Chemother. 1995, 39, 1211). To date over 250 different β-lactamases have been reported (Payne, D. J,: Du, W and Bateson, J. H. Exp. Opin. Invest. Drugs 2000, 247) and there is a need for a new generation of broad spectrum β-lactamase inhibitors. Bacterial resistance to these antibiotics could be greatly reduced by administering the β-lactam antibiotic in combination with a compound which inhibits these enzymes.

Cefepime is a parenteral aminothiazolylacetamido cephalosporin antibiotic. (Sanders, C. C. 1993. Cefepime: the next generation? Clin. Infect. Dis. 17:369-379). Even though cefepime was shown to have good activity against many pathogens that cause nosocomial pneumonia and other serious infections, it is not active against Enterococcus faecalis, Clostridium difficile and methicillin-resistant S. aureus. (Jones, R. N. 2001. Resistance patterns among nosocomial pathogens: trends over the past few years. Chest 119:397S-404S; Okamoto, M. P., R. K. Nakahiro, A. Chin, A. Bedikian, and M. V. Gill. 1994. Cefepime: a new fourth-generation cephalosporin. Am. J. Hosp. Pharm. 41:463-477.) Cefepime is also hydrolyzed by the extended-spectrum beta-lactamases (ESBLs) produced by some members of the Enterobacteriaceae.

The commercially available β-lactamase inhibitors such as clavulanic acid, sulbactam and tazobactam are all effective against Class-A producing pathogens. Clavulanic acid is clinically used in combination with amoxicillin and ticarcillin; similarly sulbactam with ampicillin and tazobactam with piperacillin. However, these compounds are ineffective against Class-C producing organisms. The mechanism of inactivation of Class-A β-lactamases (such as PCI and TEM-1) has been elucidated. (Bush, K.; Antimicrob. Agents Chemother. 1993, 37, 851; Yang, Y.; Janota, K.; Tabei, K.; Huang, N.; Seigal, M. M.; Lin, Y. I.; Rasmussen, B. A. and Shlaes, D. M. J. Biol. Chem. 2000, 35, 26674-26682).

Recently it has been shown that 6-methylidene derivatives of general formula (II) are effective, broad-spectrum β-lactamase inhibitors when combined with certain β-lactam antibiotics. WO 03/093280 A1, WO 03/093279 A1, WO 03/093277 A1 and US. 2004 132708 A1. 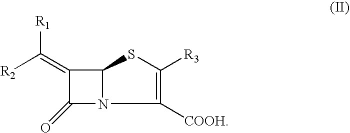
However, there remains a need for effective treatments against life threatening bacterial infections. The present invention is directed to these and other important ends.

### DETAILED DESCRIPTION OF THE INVENTION

The present invention relates to low molecular weight broad spectrum β-lactam compounds and in particular to a class of tricyclic heteroaryl substituted 6-alkylidene penems which have β-lactamase inhibitory properties when combined with a β-lactam antibiotic, including a “fourth-generation” cephalosporin antibiotic such as cefepime, a penicillin antibiotic, or a carbapenem antibiotic.

In one embodiment, the present invention relates to methods for treatin a bacterial infection or disease comprising administering to a patient in need thereof an effective amount of cefepime or a pharmaceutically acceptable salt thereof and a compound of the general formula I, as defined herein, or pharmaceutically acceptable salts or in vivo hydrolysable esters thereof. In one embodiment, the compound of general formula I is (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid sodium salt; or (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt.

In one embodiment, the present invention relates to compositions comprising a pharmaceutically acceptable carrier; cefepime or a pharmaceutically acceptable salt thereof; and a compound of the general formula I, as defined herein, or pharmaceutically acceptable salts or in vivo hydrolysable esters thereof. In one embodiment, the compound of general formula I is (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid sodium salt; or (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt.

In one embodiment, the present invention relates to use of compositions comprising cefepime or a pharmaceutically acceptable salt thereof; and a compound of the general formula I, as defined herein, or pharmaceutically acceptable salts or in vivo hydrolysable esters thereof, for the manufacture of a medicament for treating a bacterial infection or disease.

In one embodiment, the present invention relates to compounds of the general formula I, as defined herein, or pharmaceutically acceptable salts or in vivo hydrolysable esters thereof, when combined with cefepime, that are useful in the treatment of antibacterial infections in a patient.

In one embodiment, the present invention relates to compounds of the general formula I, as defined herein, or pharmaceutically acceptable salts or in vivo hydrolysable esters thereof, when combined with a β-lactam antibiotic, including a cephalosporin antibiotic, a penicillin antibiotic, or a carbapenem antibiotic, that are useful in the treatment of antibacterial infections in a patient.

In one embodiment, the present invention relates to a package comprising a pharmaceutically acceptable carrier, cefepime or a pharmaceutically acceptable salt thereof, a compound of formula I, as defined herein, or a pharmaceutically acceptable salt or in vivo hydrolysable ester thereof; and instructions, wherein the instructions comprise instructions for treating a baterial infection or disease.

In one embodiment, the present invention relates to a product comprising cefepime or a pharmaceutically acceptable salt thereof and a compound of formula I, as defined herein, or a pharmaceutically acceptable salt or in vivo hydrolysable ester thereof, as a combined preparation for separate, simultaneous or sequential administration for treating a bacterial infection or disease.

In one embodiment, the present invention relates to use of cefepime or a pharmaceutically acceptable salt thereof and a compound of formula I, as defined herein, or a pharmaceutically acceptable salt or in vivo hydrolysable ester thereof, in the preparation of a medicament for treating a bacterial infection or disease. In another embodiment, the present invention of a compound of formula I and a β-lactam antibiotic is further combined with other compounds, including, but not limited to, a dehydropeptidase (DHP) inhibitor, for example, cilastatin, that is useful in the treatment of antibacterial infections in a patient.

Chemical Definitions

As used herein, R₁ is H, optionally substituted —C1-C6 alkyl, optionally substituted -aryl, optionally substituted -heteroaryl or mono or bicyclic saturated heterocycles, optionally substituted —C3-C7 cycloalkyl, optionally substituted —C3-C6 alkenyl, optionally substituted —C3-C6 alkynyl with the proviso that both the double bond and the triple bond should not be present at the carbon atom which is directly linked to N; optionally substituted —C1-C6 per fluoro alkyl, —S(O)ₚ optionally substituted alkyl or aryl where p is 2, optionally substituted —C═Oheteroaryl, optionally substituted —C═Oaryl, optionally substituted —C═O (C1-C6) alkyl, optionally substituted —C═O (C3-C6) cycloalkyl, optionally substituted —C═O mono or bicyclic saturated heterocycles, optionally substituted C1-C6 alkyl aryl, optionally substituted C1-C6 alkyl heteroaryl, optionally substituted aryl-C1-C6 alkyl, optionally substituted heteroaryl-C1-C6 alkyl, optionally substituted C1-C6 alkyl mono or bicyclic saturated heterocycles, optionally substituted arylalkenyl of 8 to 16 carbon atoms, —CONR₆R₇, —SO₂NR₆R₇, optionally substituted arylalkyloxyalkyl, optionally substituted -alkyl-O-alkyl-aryl, optionally substituted -alkyl-O-alkyl-heteroaryl, optionally substituted aryloxyalkyl, optionally substituted heteroaryloxyalkyl, optionally substituted aryloxyaryl, optionally substituted aryloxyheteroaryl, optionally substituted C1-C6alkyl aryloxyaryl, optionally substituted C1-C6 alkyl aryloxyheteroaryl, optionally substituted alkyl aryloxy alkylamines, optionally substituted alkoxy carbonyl, optionally substituted aryloxy carbonyl, optionally substituted heteroaryloxy carbonyl. In one embodiment, R₁ is H, optionally substituted alkyl, optionally substituted aryl, —C═O(C1-C6)alkyl, C3-C6alkenyl, C3-C6alkynyl, optionally substituted cycloalkyl, SO₂alkyl, SO₂aryl, optionally substituted heterocycles, —CONR₆R₇, or optionally substituted heteroaryl.

R₂ is hydrogen, optionally substituted C1-C6 alkyl, optionally substituted C2-C6 alkenyl having 1 to 2 double bonds, optionally substituted C2-C6 alkynyl having 1 to 2 triple bonds, halogen, cyano, N—R₆R₇, optionally substituted C1-C6 alkoxy, hydroxy; optionally substituted aryl, optionally substituted heteroaryl, COOR₆, optionally substituted alkyl aryloxy alkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylene dioxy, optionally substituted aryloxy-C1-C6 alkyl amine, C1-C6 perfluoro alkyl, S(O)_{q}-optionally substituted C1-C6 akyl, S(O)_{q}-optionally substituted aryl where q is 0, 1 or 2, CONR₆R₇, guanidino or cyclic guanidino, optionally substituted C1-C6 alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, optionally substituted alkylaryloxyalkylamines, optionally substituted C3-C7 cycloalkyl, optionally substituted C3-C7 saturated or partially saturated heterocycle. In one embodiment, R₂ is H, optionally substituted alkyl, optionally substituted alkoxy, optionally substituted heteroaryl, halogen, CN, hydroxy, optionally substituted heterocycle, —CONR₆R₇, COOR₆, optionally substituted aryl, S(O)_{q}-alkyl, or S(O)_{q}-aryl.

R₃ is hydrogen, C1-C6 alkyl, C3-C6 cycloalkyl, optionally substituted aryl, optionally substituted heteroaryl. In one embodiment, R₃ is H or C1-C6 alkyl.

R₄ is H, optionally substituted C1-C6 alkyl, one of R₄ is OH, C₁-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S═(O)n (where n=0 to 2), N—R₁; in one embodiment, R₄ is H, C1-C6 alkyl, NR₆R₇, or R₄R₄ together with the carbon to which they are attached forming a spiro system of five to eight members.

R₆ and R₇ are independently H, optionally substituted C1-C6 alkyl, optionally substituted aryl, optionally substituted heteroaryl, optionally substituted C1-C6 alkyl aryl, optionally substituted arylalkyl, optionally substituted heteroarylalkyl, optionally substituted C1-C6 alkyl heteroaryl, R₆ and R₇ can together with the nitrogen to which they are attached form a 3-7 membered saturated ring system optionally having one or two heteroatoms such as N—R₁, O, S═(O)ₙ n=0-2. In one embodiment, R₆ and R₇ groups are independently H, C1-C6 alkyl, arylalkyl, heteroarylalkyl, or R₆ and R₇ together with the nitrogen to which they are attached form a 3-7 membered saturated ring system.

The term alkyl refers to both straight and branched chain alkyl moieties of 1-12 carbons unless specified otherwise; in one embodiment, of 1-8 carbon atoms; in one embodiment, of 1-6 carbon atoms; and in one embodiment, of 1-4 carbon atoms. Representative (C₁-C₆)-alkyl groups include, but are not limited to, methyl, ethyl, propyl, isopropyl, butyl, sec-butyl, tert-butyl, pentyl, isopentyl, neopentyl, hexyl, isohexyl, and neohexyl.

The term cycloalkyl refers to an alicyclic hydrocarbon group having 3-7 carbon atoms unless specified otherwise; in one embodiment, 7 carbon atoms; in one embodiment, 6 carbon atoms; in one embodiment, 5 carbon atoms; in one embodiment, 4 carbon atoms; and in one embodiment, 3 carbon atoms.

Aryl refers to an aromatic hydrocarbon moiety, e.g., 6-14 carbon atoms, for example selected from the group: phenyl, α-naphthyl, β-naphthyl, biphenyl, anthryl, tetrahydronaphthyl, fluorenyl, indanyl, biphenylenyl, acenaphthenyl, groups. In one embodiment, the aryl group is phenyl or biphenyl.

Heteroaryl refers to a aromatic heterocyclic ring system (monocyclic or bicyclic), e.g., having 5-10 ring members and 1-3 heteroatoms selected from O, N, and S, for example, where the heteroaryl moieties are selected from: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline; (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. In one embodiment, the heteroaryl group is furan, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, quinoline, isoquinoline, or naphthyridine.

The term fused tricyclic heteroaryl group refers to a group comprising three fused rings in which at least one ring has aromatic character (i.e. meets Huckel's rule (4n+2)). The fused tricyclic heteroaryl group contains 1-6 heteroatoms selected from the group consisting of O, S, N, and N—R₁. The fused tricyclic heteroaryl can be bonded through a carbon in one of the at least one aromatic rings to the remainder of the compound of formula I. The fused tricyclic heteroaryl group can contain 1-3 aromatic rings and 0-2 non-aromatic rings.

Each aromatic ring(s) in the fused tricyclic heteroaryl group may contain 5 to 7 ring atoms (including the bridgehead atoms) selected from CR₂, O, S, N, and N—R₁. Each of the aromatic ring(s) of the fused tricyclic heteroaryl group may contain 0 to 3 heteroatoms selected from O, S, N or N—R₁. The non-aromatic ring(s), if any, of the fused tricyclic heteroaryl group may contain 5-8 ring atoms (including bridgehead atoms) and contain 0-4 heteroatoms selected from N, N—R₁, O or S(O)ₙ, wherein n is 0-2. In each non-aromatic ring of the fused tricyclic heteroaryl group, one or two of the non-bridgehead carbon atoms may each be optionally substituted with one or two R₄, and each R₄ may be independently the same or different. Examples of fused tricyclic heteroaryl are optionally substituted ring systems such as imidazo[2,1-b][1,3]benzothiazole optionally substituted e.g., by for example C1-C6alkyl, C1-C6alkoxy or halo (such as chlorine or fluorine); imidazo[1,2-a]quinoline; 6,7-dihydro-5H-cyclopenta[d]imidazo[2,1-b][1,3]thiazole; imidazo[1,2-a]quinoxaline; 5,6,7,8-tetrahydro-[1,2,4]triazolo[1,5-a]pyridine dibenzo[b,f][1,4]-oxazepin-11(10H)-one optionally substituted e.g., by arylalkyl such as benzyl; 7,8-dihydro-6H-3,4,8b-triaza-as-indacene optionally substituted by C1-C6 alkoxy; 4H,10H-pyrazolo[5,1-c][1,4]benzoxazepine optionally substituted e.g., by C1-C6 alkoxy; 5H-imidazo[2,1-a]isoindole; 5,8-dihydro-6H-imidazo[2,1-b]pyrano[4,3-d][1,3]thiazole; imidazo[2,1-b]benzothiazole; [1,3]thiazolo[3,2-a]benzimidazole; 7,8-dihydro-6H-cyclopenta[3,4]pyrazolo[5,1-b][1,3]thiazole; 5,6,7,8-tetrahydroimidazo[2,1-b][1,3]-benzothiazole; 9H-imidazo[1,2-a]benzimidazole optionally substituted e.g., by C1-C6alkyl; 4H-thieno[2′,3′:4,5]thiopyrano[2,3-b]pyridine; 7,8-dihydro-6H-cyclopenta[e][1,2,4]-triazolo[1,5-a]pyrimidine optionally substituted e.g., by C1-C6alkyl; 6,7,8,9-tetrahydropyrido[3,4-e][1,2,4]triazolo[1,5-a]pyrimidine optionally substituted e.g., by C2-C7alkoxycarbonyl; 8′,9′-dihydro-6′H-spiro[1,3-dioxolane-2,7′-[1,2,4]triazolo[1,5-a]-quinazoline; 6,7,8,9-tetrahydro[1,2,4]triazolo[1,5-a]quinazoline optionally substituted e.g., by C1-C6alkyl; 7,8-dihydro-6H-cyclopenta[e]imidazo[1,2-a]pyrimidine optionally substituted e.g., by C1-C6alkoxy; 7,8-dihydro-6H-cyclopenta[e]imidazo[1,2-a]pyrimidinyl optionally substituted e.g., by arylalkyloxyalkyloxy; 3-dihydro[1,3]thiazolo[3,2-a]-benzimidazole; 2,3-dihydro[1,3]thiazolo[3,2-a]benzimidazole; 4-dihydro-2H-[1,3]thiazino[3,2-a]-benzimidazole; [1,3]thiazolo[3,2-a]benzimidazole; 7,8-dihydro-5H-pyrano[4,3-d]pyrazolo[5,1-b][1,3]-oxazole; 5,6,7,8-tetrahydropyrazolo[5,1-b][1,3]benzoxazole; and 5,6,7,8-tetrahydropyrazolo[5′,1′:2,3][1,3]oxazolo[5,4-c]pyridine optionally substituted e.g., by C2-C7alkoxycarbonyl.

A fused tricyclic heteroaryl group includes optionally substituted ring systems such as 6,7-dihydro-5H-cyclopenta[d]imidazo[2,1-b][1,3]thiazole; and 5,8-dihydro-6H-imidazo[2.1-b]pyrano[4,3-d][1,3]thiazole moieties.

If any group is said to be ‘optionally substituted’, such as for example aryl or heteroaryl, then, one or two of the following are possible substituents (the same or different): nitro, -aryl, -heteroaryl, alkoxycarbonyl-, -alkoxy, -alkoxy-alkyl, alkyl-O—C2-C4alkyl-O—, -cyano, -halogen, -hydroxy, —N—R₆R₇, -trifluoromethyl, -trifluoromethoxy, arylalkyl, alkylaryl, R₆R₇N-alkyl-, HO—C1-C6-alkyl-, alkoxyalkyl-, alkyl-S—, —SO₂N—R—₆R₇, —SO₂NHR₆, —CO₂H, CONR₆R₇, aryl-O—, heteroaryl-O—, —S(O)ₛ-aryl (where s=0-2), -alkyl-O-alkyl-NR₆R₇, -alkyl-aryl-O-alkylN-R₆R₇, C1-C6alkyl, alkenyl, alkynyl, cycloalkyl, alkoxy-alkyl-O—, R₆R₇N-alkyl-, and —S(O)ₛ-heteroaryl (where s=0-2). In one embodiment, substituents, e.g., for aryl and heteroaryl include: alkyl, halogen, —N—R₆R₇, trifluoromethyl, -trifluoromethoxy, arylalkyl, and alkylaryl.

Arylalkyl refers to Aryl-C1-C6alkyl-; Arylalkyl moieties include benzyl, 1-phenylethyl, 2-phenylethyl, 3-phenylpropyl, 2-phenylpropyl and the like. The term ‘optionally substituted’ refers to unsubstituted or substituted with 1 or 2 substituents on the alkyl or aryl moiety as defined above.

Alkylaryl refers to C1-C6alkyl-aryl-. The term ‘optionally substituted’ refers to unsubstituted or substituted with 1 or 2 substituents on the aryl or alkyl moiety as defined above.

Heteroaryl-C1-C6-alkyl refers to a heteroaryl substituted alkyl moiety wherein the alkyl chain is 1-6 carbon atoms (straight or branched). Alkyl heteroaryl moieties include Heteroaryl-(CH₂)₁₋₆— and the like. The term ‘optionally substituted’ refers to unsubstituted or substituted with 1 or 2 substituents on the alkyl or heteroaryl moiety as defined above.

C1-C6 alkylheteroaryl refers to an alkyl chain of 1-6 carbon atoms (straight or branched) attached to a heteroaryl moiety, which is bonded to the rest of the molecule. For example C1-C6-alkyl-Heteroaryl-. The term ‘optionally substituted’ refers to unsubstituted or substituted with 1 or 2 substituents on the alkyl or heteroaryl moiety as defined above.

Saturated or partially saturated heterocycles groups refers to heterocyclic rings selected from the moieties; aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. In one embodiment, saturated or partially saturated heterocycles include: aziridinyl, azetidinyl, 1,4-dioxanyl, hexahydroazepinyl, piperazinyl, piperidinyl, pyrrolidinyl, morpholinyl, thiomorpholinyl, tetrahydroquinolinyl, tetrahydroisoquinolinyl, dihydroimidazolyl, and dihydroisooxazolyl.

C1-C6 alkyl mono or bicyclic saturated or partially saturated heterocycles refers to an alkyl group (straight or branched) of C1-C6 attached to a heterocycle (which is defined before) through a carbon atom or a nitrogen atom and the other end of the alkyl chain attached to the rest of the molecule. The term ‘optionally substituted’ refers to unsubstituted or substituted with 1 or 2 substituents present on the alkyl or heterocyclic portion of the molecule, as defined before.

Arylalkyloxyalkyl refers to Aryl-C1-C6alkyl-O—C1-C6alkyl-.The term ‘optionally substituted’ refers to unsubstituted or substituted with 1 or 2 substituents present on the alkyl and/or aryl portions as defined before.

Alkyloxyalkyl refers to C1-C6 alkyl-O—C1-C6alkyl-. The term ‘optionally substituted’ refers to unsubstituted or substituted with 1 or 2 substituents present at the alkyl moiety as defined before.

Aryloxyalkyl is defined as Aryl-O—C1-C6 alkyl-. The term ‘optionally substituted’ refers to unsubstituted or substituted with 1 or 2 substituents present at the alkyl or aryl moiety as defined before.

Heteroarylalkyloxyalkyl refers to Heteroaryl-C1-C6alkyl-O—C1-C6alkyl-. The term ‘optionally substituted’ refers to unsubstituted or substituted with 1 or 2 substituents present on the alkyl or heteroaryl moiety as defined before.

Aryloxyaryl refers to Aryl-O-Aryl-. The term ‘optionally substituted’ refers to unsubstituted or substituted with 1 or 2 substituents present on the aryl moiety as defined before.

Aryloxyheteroaryl refers to Aryl-O-Heteroaryl- or -Aryl-O-Heteroaryl. In this definition either the aryl moiety or the heteroaryl moiety can be attached to the remaining portion of the molecule. The term ‘optionally substituted’ refers to unsubstituted or substituted with 1 or 2 substituents present on the aryl moiety or on the heteroaryl moiety as defined before.

Alkyl aryloxyaryl refers to Aryl-O-Aryl-C1-C6alkyl-. The term ‘optionally substituted’ refers to unsubstituted or substituted with1 or 2 substituents present at the aryl moiety as defined before.

Alkylaryloxyheteroaryl refers to Heteroaryl-O-Aryl-C1-C6alkyl-. The term ‘optionally substituted’ refers to unsubstituted or substituted with 1 or 2 substituents present on the aryl moiety or on the hetroaryl moiety as defined before.

Alkylaryloxyalkylamine refers to R₆R₇N—C1-C6alkyl-O-Aryl-C1C6alkyl-. The terms ‘optionally substituted’ refers to unsubstituted or substituted with 1 or 2 substituents present on the alkyl or aryl moiety as defined before; R₆ and R₇ as defined before.

Alkoxycarbonyl refers to C1-C6alkyl-O—C═O—. The term ‘optionally substituted’ refers to unsubstituted or substituted with 1 or 2 substituents present on the alkyl portion of the alkoxy moiety as defined before.

Aryloxycarbonyl refers to Aryl-O—C═O—. The term ‘optionally substituted’ refers to unsubstituted or substituted with 1 or 2 substituents present at the aryl moiety as defined before.

Heteroaryloxy carbonyl refers to Heteroaryl-O—C═O—. The term —optionally substituted’ refers to unsubstituted or substituted with 1 or 2 substituents present at the heteroaryl moiety as defined before.

Alkoxy refers to C1-C6alkyl-O—. The terms ‘optionally substituted’ refers to unsubstituted or substituted with 1 or 2 substituents present at the alkyl moiety as defined before.

Aryloxy refers to Aryl-O—. The term ‘optionally substituted’ refers to unsubstituted or substituted with 1 or 2 substituents present at the aryl moiety as defined before.

Heteroaryloxy refers to Heteroaryl-O—. The term ‘optionally substituted’ refers to unsubstituted or substituted with 1 or 2 substituents present at the heteroaryl moiety as defined before.

Alkenyloxy refers to C3-C6 alkene-O—. Example allyl-O—, but-2-ene-O or like moieties. The term ‘optionally substituted’ refers to unsubstituted or substituted with 1 or 2 substituents present at the alkene moiety as defined before, with the proviso that no hetero atom such as O, S or N—R₁ is present on the carbon atom, which is attached to a double bond.

Alkynyloxy refers to C3-C6alkyne-O—. Example CH≡C—CH₂—O—, or like moieties. The term ‘optionally substituted’ refers to unsubstituted or substituted with 1 or 2 substituents present at the alkyne moiety as defined before, with the proviso that no hetero atom such as O, S or N—R, is present on a carbon atom which is attached to a double or triple bond.

Alkylaminoalkoxy refers to R₆R₇N—C1-C6-alkyl-O—C1-C6-alkyl-, where the terminal alkyl group attached to the oxygen is connected to the rest of the molecule. The terms R₆ and R₇ are defined above. The term ‘optionally substituted’ refers to unsubstituted or substituted with 1 or 2 substituents present at the alkyl moiety as defined before.

Alkylenedioxy refers to —O—CH₂—O— or —O—(CH₂)₂—O—.

Aryloxyalkylamine refers to R₆R₇N—C1-C6-alkyl-O-Aryl-, where the aryl is attached to the rest of the molecule. The term ‘optionally substituted’ refers to unsubstituted or substituted with 1 or 2 substituents present at the alkyl or aryl moiety as defined before.

Arylalkenyl refers to Aryl-C2-C8alkene-, with the proviso that no hetero atom such as O, S or N—R₁ is present on the carbon atom, which is attached to a double bond. The term ‘optionally substituted’ refers to unsubstituted or substituted with 1 or 2 substituents present on the alkene or aryl moiety as defined before.

Heteroaryloxyalkyl refers to Heteroaryl-O—C1-C6alkyl-. The term ‘optionally substituted’ refers to unsubstituted or substituted with 1 or 2 substituents present at the heteroaryl moiety as defined before.

Heteroaryloxyaryl refers to Heteroaryl-O-aryl-, where the aryl moiety is attached to the rest of the molecule. The term ‘optionally substituted’ refers to unsubstituted or substituted with 1 or 2 substituents present at the heteroaryl moiety or the aryl moiety as defined before.

Alkoxy, alkoxyalkyl, alkoxyalkyloxy and alkylthioalkyloxy refers to moieties wherein the alkyl chain is 1-6 carbon atoms (straight or branched). Aryloxy, heteroaryloxy, arylthio and heteroarylthio are moieties wherein the aryl and heteroaryl groups are as herein before defined. Arylalkyloxy, heteroarylalkyloxy, arylalkylthio and heteroarylalkylthio are moieties wherein the aryl and heteroaryl groups are as herein before defined and wherein the alkyl chain is 1-6 carbons (straight or branched). Aryloxyalkyl, heteroaryloxyalkyl, aryloxyalkyloxy and heteroaryloxyalkyloxy are substituents wherein the alkyl radical is 1-6 carbon atoms. The terms monoalkylamino and dialkylamino refer to moieties with one or two alkyl groups wherein the alkyl chain is 1-6 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1-3 carbon atoms.

Pharmaceutically acceptable salts are those salts which may be administered or provided to a warm blooded animal, including sodium, potassium or calcium alkaline earth metal salts.

The term patient as used herein includes, without limitation, a human, mouse, rat, guinea pig, dog, cat, horse, cow, pig, monkey, chimpanzee, baboon, or rhesus. In one embodiment, the patient is a warm blooded animal. In another embodiment, the patient is a human.

The term effective amount as used herein refers to an amount of a compound or pharmaceutically acceptable salt of a compound that, when administered to a patient, is effective to prevent, to at least partially ameliorate, or to cure, a condition from which the patient suffers or is suspected to suffer.

The term substantially free of its corresponding opposite enantiomer as used herein means that the compound contains no more than about 10% by weight of its corresponding opposite enantiomer. In other embodiments, the compound that is substantially free of its corresponding opposite enantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term isolated and purified as used herein refers to an isolate that is separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term tautomer as used herein refers to compounds produced by the phenomenon wherein a proton of one atom of a molecule shifts to another atom. See, Jerry March, Advanced Organic Chemistry: Reactions, Mechanisms and Structures, Fourth Edition, John Wiley & Sons 1992, 69-74.

Compounds of Formula I

Compounds useful in the present invention include compounds of formula I and pharmaceutically acceptable salts or in vivo hydrolysable esters thereof: 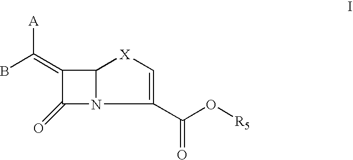
wherein:
one of A and B denotes hydrogen and the other of A and B denotes an optionally substituted fused tricyclic heteroaryl group;
X is S or O;
R₅ is hydrogen, an in vivo hydrolysable ester such as C₁-C₆ alkyl, C₅-C₆ cycloalkyl, CHR₃OCOC₁-C₆ alkyl or a salt such as Na, K, or Ca; and
R₃ is hydrogen, C₁-C₆ alkyl, C₅-C₆ cycloalkyl, optionally substituted aryl, or optionally substituted heteroaryl.

In one embodiment, X is S.

In one embodiment, R₅ is hydrogen or a salt.

In one embodiment, R₃ is hydrogen or C₁-C₆ alkyl.

In one embodiment, A denotes an optionally substituted tricyclic heteroaryl group and B denotes hydrogen.

In one embodiment, the compound of formula I has the following stereochemistry:

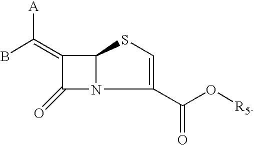

Nonlimiting examples of a tricyclic heteroaryl group include 16-A:

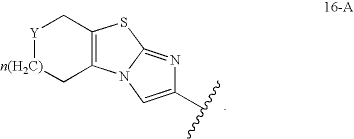

In formula 16-A, Y is O or CH₂ and n is 0 or 1. As used herein, the “ [symbol] ” designates the point of attachment of the tricyclic heteroaryl group to the rest of the molecule.

In one embodiment, when Y is O, n is 1. In another embodiment, when Y is CH₂, n is 0.

In one embodiment, the compound of formula I is: (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid sodium salt.

In one embodiment, the compound of formula I is: (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt.

In one embodiment, the compound of formula I is: (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid.

In one embodiment, the compound of formula I is: (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid.

Additional examples of optionally substituted tricyclic heteroaryl group A and B include the following:

Ring size and arrangements: (5-5-5)

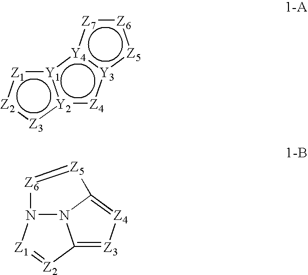

In both formula 1-A and 1-B Z₁, Z₂, Z₃, Z₄, Z₅, Z₆ and Z₇ are independently selected from CR₂, N, O, S or N—R₁ and as mentioned above one of Z₁-Z₇ is a carbon atom to which the remainder of the molecule is attached. Y₁, Y₂, Y₃ and Y₄ are independently C or N.

Ring size and arrangement: (5-5-6)

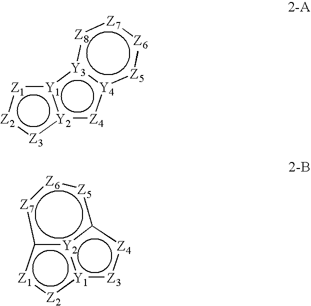

In both formula 2-A and 2-B Z₁, Z₂, Z₃, Z₄, Z₅, Z₆, Z₇ and Z₈ are independently selected from CR₂, N, O, S or N—R₁ and as mentioned above one of the Z₁-Z₈ is a carbon atom to which the remainder of the molecule is attached. Y₁, Y₂, Y₃ and Y₄ are independently C or N.

Ring size and arrangement: (5-6-5)

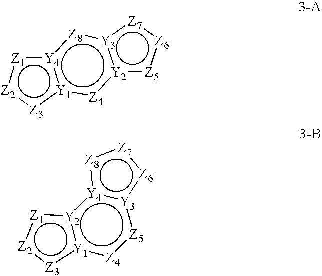

In both formula 3-A and 3-B Z₁, Z₂, Z₃, Z₄, Z₅, Z₆, Z₇ and Z₈ are independently selected from CR₂, N, O, S or N—R₁ and as mentioned above one of Z₁-Z8 is a carbon atom to which the remainder of the molecule is attached. Y₁, Y₂, Y₃ and Y₄ are independently C or N.

Ring size and arrangements: (5-6-6)

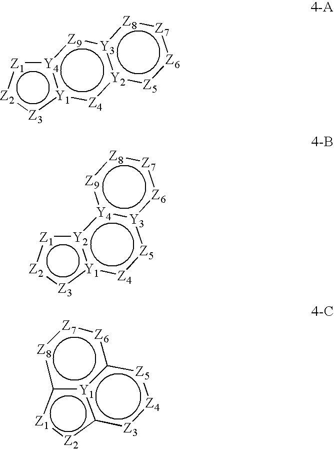

In formula 4-A, 4-B and 4-C Z₁, Z₂, Z₃, Z₄, Z₅, Z6, Z₇ and Z₈ are independently selected from CR₂, N, O, S or N—R₁ and as mentioned above one of the Z₁-Z₈ is a carbon atom to which the remainder of the molecule is attached. Y₁, Y₂, Y₃ and Y₄ are independently C or N.

Ring size and arrangements: [5-5-(non-aromatic)]

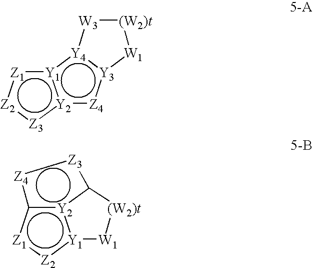

In both formula 5-A and 5-B Z₁, Z₂, Z₃ and Z₄ are independently selected from CR₂, N, O, S or N—R₁ and as mentioned above one of the Z₁-Z₄ is a carbon atom to which the remainder of the molecule is attached; and Y₁, Y₂, Y₃ and Y₄ are independently C or N. W₁, W₂ and W₃ are independently selected from CR₄R₄, S(O)ᵣ (r=0-2), O, N—R₁ with the proviso that no S—S, S—O or O—O bond formation can occur to form a saturated ring; and t=1 to 3.

Ring size and arrangement: [5-6-(non-aromatic)]

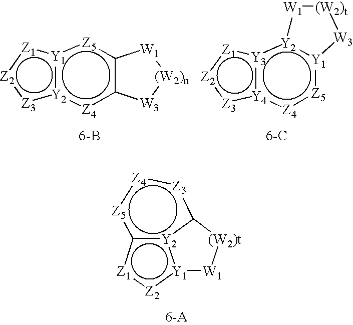

In formulae 6-A, 6-B and 6-C Z₁, Z₂, Z₃, Z₄ and Z₅ are independently selected from CR₂, N, O, S or N—R₁ and as mentioned above one of the Z₁-Z₅ is a carbon atom to which the remainder of the molecule is attached. Y₁, and Y₂ are independently C or N. W₁, W₂ and W₃ are independently CR₄R₄, S(O)ᵣ (r=0-2), O, or N—R₁ with the proviso that no S—S, S—O or O—O bond formation can occur to form a saturated ring; and t=1 to 3.

Ring size and arrangement: [5-(non-aromatic)-5]

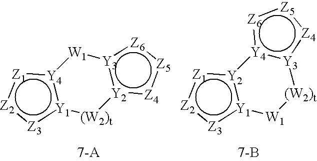

In formulae 7-A and 7-B Z₁, Z₂, Z₃, Z₄, Z₅ and Z₆ are independently selected from CR₂, N, O, S, and N—R₁; one of Z₁-Z₆ is a carbon atom to which the remainder of the molecule is attached. Y₁, Y₂, Y₃ and Y₄ are independently C or N. W₁ and W₂ are independently selected from CR₄R₄, S(O)ᵣ (r=0-2), O, N—R₁ with the proviso that no S—S, S—O or O—O bond formation can occur to form a saturated ring; and t=1 to 3.

Ring size and arrangement: [5-(non-aromatic)-6]

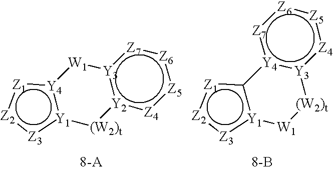

In formulae 8-A and 8-B, Z₁, Z₂, Z₃, Z₄, Z₅, Z₆ and Z₇ are independently selected from CR₂, N, O, S and N—R₁ and as mentioned above one of the Z₁-Z₇ is a carbon atom to which the remainder of the molecule is attached. Y₁, Y₂, Y₃ and Y₄ are independently C or N. W₁ and W₂ are independently CR₄R₄, S(O)ᵣ (r=0-2), O, or N—R, with the proviso that no S—S, S—O or O—O bond formation can occur to form a saturated ring; and t=0-3.

Ring size and arrangement [5-(non-aromatic)-(non-aromatic)]

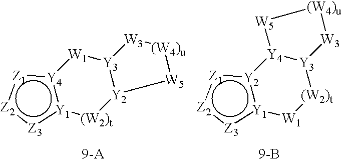

In formulae 9-A and 9-B Z₁, Z₂ and Z₃ are independently selected from CR₂, N, O, S or N—R₁; and one of Z₁-Z₃ is a carbon atom to which the remainder of the molecule is attached. Y₁ and Y₄ are independently C or N; Y₂ and Y₃ are independently CH or N; W₁, W₂, W₃, W₄ and W₅ are independently CR₄R₄, S(O)ᵣ (r=0-2), O, or N—R₁ with the proviso that no S—S, S—O or O—O bond formation can occur to form a saturated ring; t=0 to 2 and u=1 to 3.

Ring size and arrangement (6-5-6)

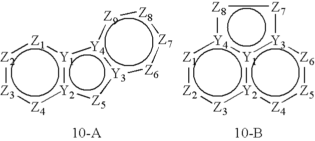

In formula 10-A and 10-B Z₁, Z₂, Z₃, Z₄, Z₅, Z₆, Z₇, Z₈ and Z₉ are independently selected from CR₂, N, O, S or N—R₁ and as mentioned above one of the Z₁-Z₉ is a carbon atom to which the remainder of the molecule is attached. Y₁, Y₂, Y₃ and Y₄ are independently C or N.

Ring size and arrangement (6-6-6)

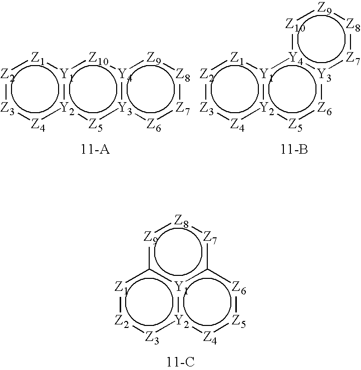

In formula 11-A, 11-B and 11-C Z₁, Z₂, Z₃, Z₄, Z₅, Z₆, Z₇, Z₈, Z₉ and Z₁₀ are independently CR₂, N, O, S or N—R₁; one of the Z₁-Z₁₀ is a carbon atom to which the remainder of the molecule is attached. Y₁, Y₂, Y₃ and Y₄ are independently C or N.

Ring size and arrangement [6-5-(non-aromatic)]

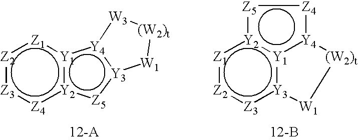

In formula 12-A and 12-B Z₁, Z₂, Z₃, Z₄ and Z₅ are independently CR₂, N, O, S or N—R₁ with the proviso that one of Z₁-Z₅ is a carbon atom to which the remainder of the molecule is attached. Y₁, Y₂, Y₃ and Y₄ are independently C or N; W₁, W₂, W₃ are independently CR₄R₄O, N—R₁, or S═(O)ᵣ (r=0-2) with the proviso that no S—S, S—O or O—O bond formation can occur to form a saturated ring; and t=1-4.

Ring size and arrangement [6-6-(non-aromatic)]

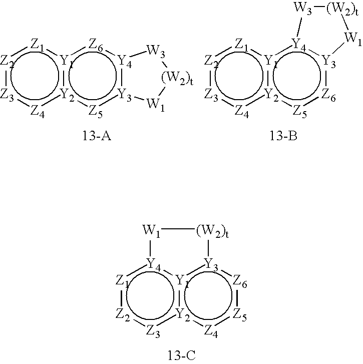

In formula 13-A, 13-B and 13-C Z₁, Z₂, Z₃, Z₄, Z₅ and Z₆ are independently CR₂, N, O, S or N—R₁; one of Z₁-Z₆ is a carbon atom to which the remainder of the molecule is attached. Y₁, Y₂, Y₃ and Y₄ are independently C or N; W₁, W₂ and W₃ are independently CR₄R₄, S(O)r (r=0-2), O, or N—R₁ with the proviso that no S—S, S—O or O—O bond formation can occur to form a saturated ring; and t=1 to 3.

Ring size and arrangement [6-(non-aromatic)-6]

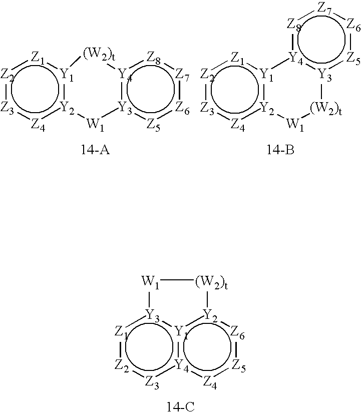

In formula 14-A, 14-B and 14-C Z₁, Z₂, Z₃, Z₄, Z₅, Z₆, Z₇ and Z₈ are independently CR₂, N, O, S or N—R₁; one of Z₁-Z₈ is a carbon atom to which the remainder of the molecule is attached. Y₁, Y₂, Y₃ and Y₄ are independently C or N; W₁, and W₂ are independently CR₄R₄, S(O)r (r=0-2), O, or N—R₁ with the proviso that no S—S, S—O or O—O bond formation can occur to form a saturated ring; and t=1 to 2.

Ring size and arrangement [6-(non-aromatic)-(non-aromatic)]

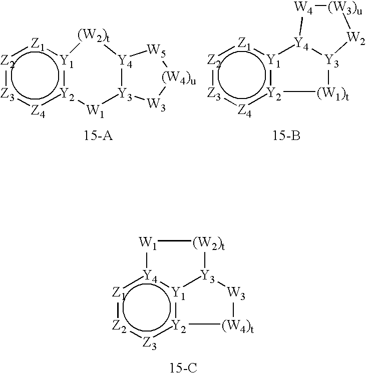

In formula 15-A, 15-B and 15-C Z₁, Z₂, Z₃and Z₄ are independently CR₂, N, O, S or N—R₁; and one of Z₁-Z₄ is a carbon atom to which the remainder of the molecule is attached. Y₁, Y₂, Y₃ and Y₄ are independently C or N; W₁, W₂, W₃, W₄ and W₅ are independently CR₄R₄, S(O)r (r=0-2), O, or N—R₁ with the proviso that no S—S, S—O or O—O bond formation can occur to form a saturated ring; t=1 to 3 and u=1 to 3.

Additional examples of optionally substituted tricyclic heteroaryl groups A and B are set forth in WO 03/093280 A1, WO 03/093277 A1 and US 2004 132708 A1.

Compounds useful in the present invention include pharmaceutically acceptable salts or in vivo hydrolysable esters thereof, and as such, the term “compound” as used herein includes a pharmaceutically acceptable salt or in vivo hydrolysable ester thereof. A compound's structural formula also includes any tautomers, any stereoisomers (except where stereochemistry is clearly noted) and any crystalline forms.

The compounds of formula I can contain an asymmetric carbon atom and some of the compounds of formula I can contain one or more asymmetric centers, and can thus give rise to optical isomers and diastereomers. While in some cases depicted without respect to stereochemistry in the compounds of formula I, the present invention includes such optical isomers and diastereomers, as well as racemic and resolved, enantiomerically pure R and S stereoisomers, and also other mixtures of the R and S stereoisomers and pharmaceutically acceptable salts thereof. Where a stereoisomer is provided, it can in some embodiments be provided substantially free of its corresponding opposite enantiomer.

In addition, the compounds of formula I can exist as tautomers. Such tautomers can be transient or isolatable as a stable product. These tautomers are within the scope of the present invention.

Prodrugs of the compounds of formula I are also within the scope of the present invention.

Methods of Making Compounds of Formula I

The compounds of formula I can be prepared using a variety of methods starting from commercially available compounds, known compounds, or compounds prepared by known methods. General synthetic routes to many of the compounds are included in the following schemes. It is understood by those skilled in the art that protection and deprotection steps not shown in the Schemes may be required for these syntheses, and that the order of steps may be changed to accommodate functionality in the target molecule.

For example, compounds of formula I can be synthesized according to the procedures outlined in WO 03/093280 A1, WO 03/093277 A1 and US 2004 132708 A1.

Therapeutic Administration

In one embodiment, a compound of formula I has β-lactamase inhibitory and antibacterial properties and is useful for the treatment of infections in a patient when combined with cefepime. In one embodiment of the present invention, a compound of formula I in combination with cefepime provides an effective treatment of a bacterial infection or disease.

In one embodiment, a compound of formula I has β-lactamase inhibitory and antibacterial properties and is useful for the treatment of infections in a patient when combined with a β-lactam antibiotic. In one embodiment of the present invention, a compound of formula I in combination with a β-lactam antibiotic provides an effective treatment of a bacterial infection or disease.

β-lactam antibiotics as used herein include penicillin antibiotics, cephalosporin antibiotics, and carbapenem antibiotics. For example, penicillin antibiotics such as carbenicillin, azlocillin, mezlocillin, mecillinam, nafcillin, and oxacillin; cephalosporin antibiotics such as cefaclor, cefamandol, cefdinir, cefditoren, cefatamet, cefixime, cefmetazole, cefotaxime, cefotetan, cefoxitin, cefpodoxime, ceftibuten, ceftizoxime, and cefuroxime; and carbapenem antibiotics such as loracarbef, imipenem, meropenem, and ertapenem; are useful for the treatment of infections in a patient when combined with a compound of formula I.

In one embodiment, a compound of formula I when used in combination with cefepime results in increased antibacterial activity (synergistic effect) against a Class-A producing organism. In one embodiment, a compound of formula I when used in combination with cefepime results in increased antibacterial activity (synergistic effect) against a Class-B producing organism. In one embodiment, a compound of formula I when used in combination with cefepime results in increased antibacterial activity (synergistic effect) against a Class-C producing organism. In one embodiment, a compound of formula I when used in combination with cefepime results in increased antibacterial activity (synergistic effect) against a Class-D producing organism. In another embodiment, a compound of formula I when used in combination with cefepime results in increased antibacterial activity (synergistic effect) against a Class-A and a Class-C producing organism. In still another embodiment, a compound of formula I when used in combination with cefepime results in increased antibacterial activity (synergistic effect) against a Class-A, a Class-C, and a Class-D producing organism.

In one embodiment, a compound of formula I when used in combination with a β-lactam antibiotic results in increased antibacterial activity (synergistic effect) against a Class-A producing organism. In one embodiment, a compound of formula I when used in combination with a β-lactam antibiotic results in increased antibacterial activity (synergistic effect) against a Class-B producing organism. In one embodiment, a compound of formula I when used in combination with a β-lactam antibiotic results in increased antibacterial activity (synergistic effect) against a Class-C producing organism. In one embodiment, a compound of formula I when used in combination with a β-lactam antibiotic results in increased antibacterial activity (synergistic effect) against a Class-D producing organism. In another embodiment, a compound of formula I when used in combination with a β-lactam antibiotic results in increased antibacterial activity (synergistic effect) against a Class-A and a Class-C producing organism. In still another embodiment, a compound of formula I when used in combination with a β-lactam antibiotic results in increased antibacterial activity (synergistic effect) against a Class-A, a Class-C, and a Class-D producing organism.

In one embodiment, administration of the compounds of formula I is provided in conjunction with prior, simultaneous or subsequent administration of cefepime (“co-administration”). “Provided” includes direct administration as well as in vivo, e.g. pro-drugs. When the compounds of formula I are co-administered with cefepime, the ratio of the amount of the compound to the amount of the cefepime may vary in a wide range. The ratio of cefepime to the compound of formula I may vary from 1:1 to 100:1. In one embodiment, the ratio of cefepime to the compound of formula I is less than 10:1.

In one embodiment, administration of the compounds of formula I is provided in conjunction with prior, simultaneous or subsequent administration of a β-lactam antibiotic (“co-administration”). When the compounds of formula I are co-administered with a β-lactam antibiotic, the ratio of the amount of the compound to the amount of the cefepime may vary in a wide range. The ratio of a β-lactam antibiotic to the compound of formula I may vary from 1:1 to 100:1. In one embodiment, the ratio of a β-lactam antibiotic to the compound of formula I is less than 10:1.

In one embodiment, the compositions of the present invention are in a form suitable for oral (PO), intravenous (IV) or topical administration. In one embodiment, the compositions of the invention are in a form of tablets, capsules, creams, syrups, suspension, sterile solutions suitable for injection or infusion.

In one embodiment, a compound of formula I and cefepime are administered in doses commonly employed when such agents are used individually for the treatment of a bacterial infection or disease.

In one embodiment, a compound of formula I and a β-lactam antibiotic are administered in doses commonly employed when such agents are used individually for the treatment of a bacterial infection or disease.

In another embodiment, a compound of formula I and cefepime act synergistically and are administered in doses that are less than the doses commonly employed when such agents are used individually for the treatment of a bacterial infection or disease.

In another embodiment, a compound of formula I and a β-lactam antibiotic act synergistically and are administered in doses that are less than the doses commonly employed when such agents are used individually for the treatment of a bacterial infection or disease.

As used herein, cefepime includes a pharmaceutically acceptable salt thereof.

Cefepime can be administered to a patient at a dosage ranging from about 250 mg to about 2 g per administration. In one embodiment, the dosage of cefepime is about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.1 g, about 1.2 g, about 1.25 g, about 1.3 g, about 1.4 g, about 1.5 g, about 1.6 g, about 1.7 g, about 1.75 g, about 1.8 g, or about 1.9 g. Cefepime can be administered at a time ranging from every 8 h to every 48 hr. In one embodiment, cefepime is administered every 12 h, every 16 h, every 20 h, every 24 h, every 28 h, every 32 h, every 36 h, every 40, or every 44 h. Cefepime can be administered for a duration ranging from about 7 days to about 10 days. In a specific embodiment, cefepime is administered for about 8 days or about 9 days.

As used herein, a β-lactam antibiotic includes a pharmaceutically acceptable salt thereof.

When administered to a patient, a compound (e.g., a compound of formula I, cefepime, or a β-lactam antibiotic) can be administered neat or as a component of a composition that comprises a physiologically acceptable carrier or vehicle. A composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

In one embodiment, the invention provides a composition comprising cefepime or a pharmaceutically acceptable salt thereof and a compound of formula I or a pharmaceutically acceptable salt or in vivo hydrolysable ester thereof. In another embodiment, the invention provides a composition comprising a compound of formula I or a pharmaceutically acceptable salt or in vivo hydrolysable ester thereof, and a composition comprising cefepime or a pharmaceutically acceptable salt thereof.

In one embodiment, the invention provides a composition comprising a β-lactam antibiotic or a pharmaceutically acceptable salt thereof and a compound of formula I or a pharmaceutically acceptable salt or in vivo hydrolysable ester thereof. In another embodiment, the invention provides a composition comprising a compound of formula I or a pharmaceutically acceptable salt or in vivo hydrolysable ester thereof, and a composition comprising a β-lactam antibiotic or a pharmaceutically acceptable salt thereof.

The present compositions, comprising compounds or pharmaceutically acceptable salts of compounds can be administered orally. The compositions of the invention can also be administered by any other convenient route, for example, by continuous infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in release of the compound or a pharmaceutically acceptable salt of the compound into the bloodstream. The mode of administration is left to the discretion of the practitioner.

In one embodiment, the compound or a pharmaceutically acceptable salt of the compound of formula I is administered orally.

In one embodiment, cefepime is administered orally.

In one embodiment, the β-lactam antibiotic is administered orally.

In another embodiment, the compound or a pharmaceutically acceptable salt of the compound of formula I is administered intravenously.

In one embodiment, cefepime is administered intravenously.

In one embodiment, the β-lactam antibiotic is administered intravenously.

In another embodiment, it may be desirable to administer the compound or a pharmaceutically acceptable salt of the compound of formula I locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compound or a pharmaceutically acceptable salt of the compound of formula I into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound or a pharmaceutically acceptable salt of the compound of formula I can be formulated as a suppository, with traditional binders and excipients such as triglycerides.

In another embodiment, the compound or a pharmaceutically acceptable salt of the compound of formula I can be delivered in a vesicle, in particular a liposome (see Langer, Science 1990, 249,1527-1533 and Treat et al., Liposomes in the Therapy of Infectious Disease and Cancer 1989, 317-327 and 353-365).

In yet another embodiment, the compound or a pharmaceutically acceptable salt of the compound of formula I can be delivered in a controlled-release system or sustained-release system (see, e.g., Goodson, in Medical Applications of Controlled Release, vol. 2, 1984, 115-138). Other controlled or sustained-release systems discussed in the review by Langer, Science 1990, 249, 1527 1533 can be used. In one embodiment, a pump can be used (Langer, Science 1990, 249, 1527-1533; Sefton, CRC Crit. Ref. Biomed. Eng. 1987, 14, 201; Buchwald et al., Surgery 1980, 88, 507; and Saudek et al., N. Engl. J Med. 1989, 321, 574). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 1983 2, 61; Levy et al., Science 1935, 228, 190; During et al., Ann. Neural. 1989, 25, 351; and Howard et al., J. Neurosurg. 1989, 71, 105).

In yet another embodiment, a controlled- or sustained-release system can be placed in proximity of a target of the compound or a pharmaceutically acceptable salt of the compound of formula I, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a patient. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The compound or pharmaceutically acceptable salt of the compound of formula I can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences 1447 1676 (Alfonso R. Gennaro, ed., 19th ed. 1995).

In one embodiment, the compound or a pharmaceutically acceptable salt of the compound of formula I is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of the compounds or pharmaceutically acceptable salts of the compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment, the compound or a pharmaceutically acceptable salt of the compound of formula I can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the compound or a pharmaceutically acceptable salt of the compound of formula I is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In another embodiment, the compound or pharmaceutically acceptable salt of the compound of formula I can be administered transdermally through the use of a transdermal patch. Transdermal administrations include administrations across the surface of the body and the inner linings of the bodily passages including epithelial and mucosal tissues. Such administrations can be carried out using the present compounds or pharmaceutically acceptable salts of the compounds, in lotions, creams, foams, patches, suspensions, solutions, and suppositories (e.g., rectal or vaginal).

Transdermal administration can be accomplished through the use of a transdermal patch containing the compound or pharmaceutically acceptable salt of the compound and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The compounds or pharmaceutically acceptable salts of the compounds of formula I may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound or a pharmaceutically acceptable salt of the compound of formula I can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the compound or a pharmaceutically acceptable salt of the compound of formula I to treat or prevent a bacterial infection or disease in a minimal amount of time. Advantages of controlled- or sustained-release compositions include extended activity of the drug, reduced dosage frequency, and increased compliance by the patient being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound of formula I that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound of formula I in the body, the compound or a pharmaceutically acceptable salt of the compound of formula I can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, the present invention is directed to prodrugs of the compounds or pharmaceutically acceptable salts of compounds of formula I. Various forms of prodrugs are known in the art, for example as discussed in Bundgaard (ed.), Design of Prodrugs, Elsevier 1985; Widder et al. (ed.), Methods in Enzymology, vol. 4, Academic Press 1985; Kgrogsgaard-Larsen et al. (ed.); “Design and Application of Prodrugs”, Textbook of Drug Design and Development, 1991, Chapter 5, 113-191; Bundgaard et al., Journal of Drug Delivery Reviews, 1992, 8, 1-38; Bundgaard et al., J. Pharmaceutical Sciences, 1988, 77, 285 et seq.; and Higuchi and Stella (eds.), Prodrugs as Novel Drug Delivery Systems, American Chemical Society (1975).

The amount of the compound or a pharmaceutically acceptable salt of the compound of formula I is an amount that is effective for treating a bacterial infection or disease. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound of formula I that is effective for treating a bacterial infection or disease will typically range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The compound or a pharmaceutically acceptable salt of the compound of formula I can be assayed in vitro or in vivo for the desired therapeutic or prophylactic activity prior to use in humans. Animal model systems can be used to demonstrate safety and efficacy.

Therapeutic Uses

In one embodiment, the invention provides a method for treating a bacterial infection or disease comprising providing to a patient in need thereof an effective amount of cefepime or a pharmaceutically acceptable salt thereof and a compound of formula I or pharmaceutically acceptable salt or in vivo hydrolysable ester thereof.

In one embodiment, the invention provides a method for treating a bacterial infection or disease comprising providing to a patient in need thereof an effective amount of a β-lactam antibiotic or a pharmaceutically acceptable salt thereof and a compound of formula I or pharmaceutically acceptable salt or in vivo hydrolysable ester thereof.

In another embodiment, the method for treating a bacterial infection or disease comprises co-administering cefepime or a pharmaceutically acceptable salt thereof and the compound of formula I or pharmaceutically acceptable salt or in vivo hydrolysable ester thereof. For example, the compound of formula I can be provided in conjunction with, prior, simultaneous, or subsequent to cefepime.

In another embodiment, the method for treating a bacterial infection or disease comprises co-administering a β-lactam antibiotic or a pharmaceutically acceptable salt thereof and the compound of formula I or pharmaceutically acceptable salt or in vivo hydrolysable ester thereof. For example, the compound of formula I can be provided in conjunction with, prior, simultaneous, or subsequent to the β-lactam antibiotic.

In one embodiment, the ratio of cefepime or pharmaceutically acceptable salt thereof to the compound of formula I or pharmaceutically acceptable salt of in vivo hydrolysable ester thereof is from about 1:1 to about 100:1 (w/w).

In one embodiment, the ratio of β-lactam antibiotic or pharmaceutically acceptable salt thereof to the compound of formula I or pharmaceutically acceptable salt of in vivo hydrolysable ester thereof is from about 1:1 to about 100:1 (w/w).

In one embodiment, the ratio (w/w) of cefepime or pharmaceutically acceptable salt thereof to the compound of formula I or pharmaceutically acceptable salt or in vivo hydrolysable ester thereof is from about 1:1 to about 80:1; 1:1 to about 70:1; 1:1 to about 60:1; from about 1:1 to about 50:1; 1:1 to about 40:1; from about 1:1 to about 30:1; from about 1:1 to about 20:1; from about 1:1 to about 15:1; 1:1 to about 14:1; 1:1 to about 13:1; from about 1:1 to about 12:1; from about 1:1 to about 11:1; from about 1:1 to about 10:1; from about 1:1 to about 9:1; from about 1:1 to about 8:1; from about 1:1 to about 7:1; from about 1:1 to about 6:1; from about 1:1 to about 5:1; from about 1:1 to about 4:1; from about 1:1 to about 3:1; or from about 1:1 to about 2:1.

In one embodiment, the ratio (w/w) of P-lactam antibiotic or pharmaceutically acceptable salt thereof to the compound of formula I or pharmaceutically acceptable salt or in vivo hydrolysable ester thereof is from about 1:1 to about 80:1; 1:1 to about 70:1; 1:1 to about 60:1; from about 1:1 to about 50:1; 1:1 to about 40:1; from about 1:1 to about 30:1; from about 1:1 to about 20:1; from about 1:1 to about 15:1; 1:1 to about 14:1; 1:1 to about 13:1; from about 1:1 to about 12:1; from about 1:1 to about 11:1; from about 1:1 to about 10:1; from about 1:1 to about 9:1; from about 1:1 to about 8:1; from about 1:1 to about 7:1; from about 1:1 to about 6:1; from about 1:1 to about 5:1; from about 1:1 to about 4:1; from about 1:1 to about 3:1; or from about 1:1 to about 2:1.

In one embodiment, the ratio of the cefepime or pharmaceutically acceptable salt thereof to the compound of formula I or pharmaceutically acceptable salt or in vivo hydrolysable ester thereof is less than about 10:1 (w/w).

In one embodiment, the ratio of the β-lactam antibiotic or pharmaceutically acceptable salt thereof to the compound of formula I or pharmaceutically acceptable salt or in vivo hydrolysable ester thereof is less than about 10:1 (w/w).

In one embodiment, the methods comprise orally administering to a patient.

In another embodiment, the methods comprise intravenously administering to a patient.

In one embodiment, the methods of the present invention are useful for treating a bacterial infection or disease that is cefepime-resistant.

In one embodiment, the methods of the present invention are useful for treating a bacterial infection or disease that is β-lactam antibiotic-resistant.

In one embodiment, the methods of the present invention are useful for treating a bacterial infection or disease selected from a skin infection, a skin structure infection, an intra-abdominal infection, a diabetic foot infection, nosocomial pneumonia, hospital acquired pneumonia, or febrile neutropenia.

In one embodiment, the methods of the present invention are useful for treating a bacterial infection or disease caused by Enterobacteriaceae, non-Enterobacteriaceae gram negative rods, Pseudomonas aeruginosa, staphylococci, or streptococci.

### EXAMPLES

### Example 1

### IC₅₀ Determination for Compound of Formula I

β-Lactamase inhibitory activity of the penem inhibitors was determined spectrophotometrically as described by Bush et al., [Bush, K., Macalintal, C., Rasmussen, B. A., Lee, V. and Yang, Y. Antimicrobial Agents and Chemotherapy 1993, 37, 851]. Homogeneously purified class A β-lactamases TEM-1 from E. coli and Imi-1 from Enterobacter cloacae, class B enzyme CcrA from Bacteroides fragilis and class C enzyme AmpC from Enterobacter cloacae were employed in the assay. The enzyme concentrations for TEM-1, Imi-1, CcrA and AmpC were 4.3, 7.1, 1.2 and 2.1 nM, respectively. A wide range of inhibitor concentrations were prepared in 50 mM PO₄, pH 7.0 to include the possible IC₅₀ values. The substrate used to initiate the enzyme reaction was nitrocefin at 50 μg/ml in the same buffer as the inhibitor. Initially the enzyme and inhibitor (20 μl each) were preincubated for 10 minutes at 25° C. prior to the addition of 160 μl volume of nitrocefin. Initial rates of hydrolysis were monitored for 5 minutes at 495 nm using a Molecular Devices Spectra Max 250 with kinetic protocol of SoftMax Program. Readings from the Spectra Max 250 were exported and transferred to Microsoft Excel. The percent of inhibition of each inhibitor concentration was calculated based on the control enzyme activity. The inhibitor concentration that caused a 50% reduction in the enzymatic activity (IC50) was determined graphically.

TABLE 1
β-Lactamase Inhibition Data

IC₅₀ (nM)

| | | |
|---|---|---|
| Class-A | Class-B | Class-C |

| Compound | TEM-1 | Imi | Ccr | AmpC |
|---|---|---|---|---|
| Compound 1 | 1.4 | 72 | 240 | 2.4 |
| Compound 2 | 2.8 | 100 | 200 | 1.5 |

### Example 2

### Antimicrobial Susceptibility Testing

The in vitro activities of the antibiotics were determined by the microbroth dilution method as recommended by the National Committee for Clinical Laboratory Standards (NCCLS). (NCCLS. 2000. Methods for Dilution Antimicrobial Susceptibility Tests for Bacteria That Grow Aerobically; Approved Standards: M7-A5, vol. 19. National Committee for Clinical Laboratory Standards, Villanova, Pa.). Mueller-Hinton II broth (MHBII) (BBL Cockeysville, Md.), was used for the testing procedure. Microtiter plates containing 50 μl per well of two-fold serial dilutions of cefepime combined with a constant amount (4 μg/ml) of a β-lactamase inhibitor (final concentration) were inoculated with 50 μl of inoculum to yield the appropriate density (10⁵ CFU/ml) in 100 μl. The plates were incubated for 18-22 hours at 35 C. in ambient air. The minimal inhibitory concentration (MIC) for all isolates was defined as the lowest concentration of antimicrobial agent that completely inhibits the growth of the organism as detected by the unaided eye. The MIC data obtained by the above said procedure are listed in Table 2.

TABLE 2
Minimal Inhibitory Concentration (μg/ml) Data: Inc: 35° C. for 18 hours

| | |
|---|---|
| Method: | Broth Dilution |
| Medium: | MHBII |
| Compound: | Diluted in MHBII; 0.05 ml/well |
| Inoc.: | Prompt Inoculation System; diluted 0.1 + 9.9; 0.05 ml/well |
| Inc.: | 35° C. for 18 hr |

| | | | | Cefepime + |
|---|---|---|---|---|
| | | β-lactamase | Control | 4 ug/ml |
| | Organism (enzyme) | Class | Cefepime | Compound 1 |
| 1 | E. coli ATCC 25922 (none) | none | 0.03 | 0.03 |
| 2 | E. coli GC 2804 (imp) | none | 0.03 | 0.03 |
| 3 | E. coli GC 2844 (none) | none | 0.06 | 0.06 |
| 4 | E. coli ATCC 35218 TEM-1 | A | 0.03 | 0.03 |
| 5 | E. coli GC 2847 (TEM-1) | A | 0.12 | 0.06 |
| 6 | E. coli LSU 80-8 GC 6265 TEM-1 | A | 0.12 | 0.06 |
| 7 | A. baumannii GC 7685 TEM-1 | A | >16 | 16 |
| 8 | E. coli GC 1684 (TEM-10) | A (ESBL) | >16 | 0.06 |
| 9 | E. coli GC 1995 (TEM-10) | A (ESBL) | >16 | 0.06 |
| 10 | E. coli GC 2009 (TEM-10) | A (ESBL) | >16 | 0.50 |
| 11 | E. coli GC 2300 (TEM-28) | A (ESBL) | >16 | 0.25 |
| 12 | E. coli GC 2400 (TEM-43) | A (ESBL) | >16 | 2 |
| 13 | E. coli GC 6368 (SHV-7) | A (ESBL) | 2 | 0.03 |
| 14 | E. coli GC 2017 (TEM-10 + pl 6.3; 8.1) | A (ESBL) | 16 | 0.12 |
| 15 | E. coli GC 2021 (TEM-10 + pl 6.3) | A (ESBL) | 8 | 0.12 |
| 16 | K. pn GC 1510 (TEM-10) | A (ESBL) | 0.50 | 0.06 |
| 17 | K. pn GC 1516 (TEM-26) | A (ESBL) | >16 | 1 |
| 18 | K. pn GC 1827 (TEM-3) | A (ESBL) | 4 | 0.06 |
| 19 | K. pn GC 1830 (SHV-2) | A (ESBL) | 2 | 0.06 |
| 20 | K. pn GC 1832 (SHV-4) | A (ESBL) | 2 | 0.12 |
| 21 | K. pn GC 6639 (SHV-13) | A (ESBL) | 8 | 0.25 |
| 22 | S. typhimurium GC 4197 CTX-M-5 | A (ESBL) | >16 | 1 |
| 23 | E. coli GC 1499 (TEM-4 + TEM-1) | A, A (ESBL) | >16 | 1 |
| 24 | E. coli GC 1695 (TEM-1 + TEM-10) | A, A (ESBL) | 16 | 0.06 |
| 25 | E. coli GC 2015 (TEM-10 + SHV-1) | A, A (ESBL) | >16 | 0.25 |
| 26 | E. coli GC 2146 (TEM-1; SHV-7) | A, A (ESBL) | >16 | 0.12 |
| 27 | E. coli GC 5901 (TEM-1 and SHV-8) | A, A (ESBL) | >16 | 2 |
| 28 | E. coli GC 6260 (TEM-1 + SHV-5) | A, A (ESBL) | >16 | 0.06 |
| 29 | K. pn GC 1507 (TEM-9 + SHV-1) | A, A (ESBL) | 8 | 0.50 |
| 30 | K. pn GC 2006 (TEM-10 + SHV-1) | A, A (ESBL) | >16 | 0.25 |
| 31 | K. pn GC 6657 (TEM-1 + SHV-27) | A, A (ESBL) | 0.12 | 0.06 |
| 32 | K. pn GC 6494 (TEM-1 + SHV-5) | A, A (ESBL) | 4 | 0.12 |
| 33 | K. pn GC 3057 (CAZ-R; P/T-R) | A, A (ESBL) | 16 | 0.25 |
| 34 | K. pn GC 1554 (TEM-1 + TEM-26 + SHV-1) | A, A (ESBL) | 8 | 0.25 |
| 35 | K. pn GC 1963 (TEM-10 + SHV-1 + SHV-ESBL) | A, A (ESBL) | >16 | 0.25 |
| 36 | K. pn GC 6488 (TEM-1 + SHV-5 + SHV-7) | A, A (ESBL) | 8 | 1 |
| 37 | K. pn GC 6651 (TEM-1 + SHV-1 + SHV-5) | A, A (ESBL) | 2 | 0.25 |
| 38 | S. typhimurium GC 4198 SHV-1 CTX-M-5 | A, A (ESBL) | >16 | 0.50 |
| 39 | E. coli GC 2253 (IRT-2) | A (IRT) | 0.03 | 0.03 |
| 40 | E. coli GC 2920 (IRT-2) | A (IRT) | 0.06 | 0.06 |
| 41 | E. coli GC 2906 (Imi-1) | A (Cb'ase) | 0.50 | 0.06 |
| 42 | K. oxytoca GC 7627 TEM-1 K1 KPC-2 | A, A (Cb'ase) | >16 | 2 |
| 43 | K. pneumoniae GC 7635 SHV-1, KPC2 | A, A (Cb'ase) | >16 | 8 |
| 44 | K. pneumoniae GC 7632 TEM-1 KPC-2, SHV-7, SHV-12 | A, A (Cb'ase), | >16 | 16 |
| | | A (ESBL) | | |
| 45 | K. pneumoniae GC 7636 TEM-1 KPC-2 SHV-12 | A, A (Cb'ase), | >16 | 16 |
| | | A (ESBL) | | |
| 46 | K. pneumoniae GC 7645 TEM-30 TEM-1 KPC-2 SHV- | A, A (Cb'ase), | >16 | >16 |
| | 12 | A (ESBL), A(IRT) | | |
| 47 | E. coli GC 2805 (CcrA) | B | >16 | >16 |
| 48 | S. maltophilia GC 1712(L1) | B | >16 | 16 |
| 49 | A. baumannii GC 7684 AmpC, AmpC | C | 16 | 8 |
| 50 | A. baumannii GC 7687 AmpC | C | 16 | 16 |
| 51 | C. freundii GC 4164 Inducible AmpC | C | 2 | 0.06 |
| 52 | C. freundii GC 4187 Stably derepressed AmpC | C | 2 | 0.25 |
| 53 | C. freundii PT 1499 Stably derepressed AmpC | C | 2 | 0.12 |
| 54 | E. aerogenes 1697-MP GC 7845 AmpC | C | 0.50 | 0.06 |
| 55 | E. cloacae GC 1475 (P99) | C | 1 | 0.12 |
| 56 | E. cloacae GC 1477 (AmpC) | C | 4 | 0.25 |
| 57 | E. cloacae GC 1712 AmpC-inducible | C | 0.25 | 0.06 |
| 58 | E. cloacae GC 1713 AmpC- constitutive | C | 16 | 0.12 |
| 59 | E. cloacae GC 4142 (AmpC) | C | 8 | 0.06 |
| 60 | E. cloacae GC 6991 (AmpC) | C | 0.50 | 0.50 |
| 61 | E. cloacae PT 1494 Inducible AmpC | C | 0.25 | 0.06 |
| 62 | E. cloacae PT 197 Stably derepressed AmpC | C | 1 | 0.50 |
| 63 | E. cloacae PT 967 Inducible AmpC | C | 0.12 | 0.12 |
| 64 | E. coli GC 2894 (AmpC) | C | 2 | 0.06 |
| 65 | E. coli GC 2905 (P99) | C | 2 | 0.06 |
| 66 | E. coli GC 6539 CMY-2 | C | 2 | 0.06 |
| 67 | K. pneumoniae GC 7820 Act 1 | C | 0.50 | 0.25 |
| 68 | K. pneumoniae GC 7821 DHA 1 | C | 0.06 | 0.03 |
| 69 | K. pneumoniae GC 7822 Act 1 | C | >16 | 0.50 |
| 70 | K. pneumoniae GC 7823 Fox 5 | C | 1 | 0.12 |
| 71 | K. pneumoniae GC 7824 Fox 5 | C | 2 | 0.50 |
| 72 | S. marcescens GC 4132 (AmpC) | C | 0.50 | 0.12 |
| 73 | S. marcescens GC 4132 Stably derepressed AmpC | C | 1 | 0.12 |
| 74 | S. marcescens GC 4145 Inducible AmpC | C | 1 | 0.25 |
| 75 | S. marcescens GC 4150 Stably derepressed AmpC | C | 1 | 0.25 |
| 76 | S. marcescens PT 488 Stably derepressed AmpC | C | 1 | 0.12 |
| 77 | S. marcescens PT 6003 Stably derepressed AmpC | C | 0.50 | 0.25 |
| 78 | S. marcescens PT 696 Inducible AmpC | C | 0.50 | 0.06 |
| 79 | P. aeruginosa ATCC 27853 AmpC-inducable | C | 2 | 2 |
| 80 | P. aeruginosa GC 1763 AmpC-inducible | C | 2 | 1 |
| 81 | P. aeruginosa GC 1764 (AmpC) | C | 8 | 2 |
| 82 | P. aeruginosa GC 1764 AmpC-constitutive | C | 8 | 0.50 |
| 83 | P. aeruginosa GC 3153 Inducible AmpC | C | 16 | 8 |
| 84 | P. aeruginosa GC 4161 Stably derepressed AmpC | C | >16 | 16 |
| 85 | P. aeruginosa PT 8411 PTZ-R* | C (presumed) | 16 | 4 |
| 86 | P. aeruginosa PT 8503 PTZ-R | C (presumed) | 8 | 2 |
| 87 | P. aeruginosa PT 9025 PTZ-R | C (presumed) | >16 | 16 |
| 88 | P. aeruginosa PT 9236 PTZ-R | C (presumed) | >16 | 16 |
| 89 | P. aeruginosa PT 9587 PTZ-R | C (presumed) | 4 | 2 |
| 90 | A. baumannii GC 7692 TEM-1, AmpC | A, C | >16 | >16 |
| 91 | C. freundii GC 4171 TEM-1 + InducibleAmpC | A, C | 2 | 0.06 |
| 92 | E. coli GC 2295 TEM-1 + AmpC | A, C | 0.50 | 0.12 |
| 93 | K. pn GC 3104 (MIR-1 + TEM-1) | A, C | 1 | 0.50 |
| 94 | K. pn GC 6655 (TEM-1 + ACT-1) | A, C | 1 | 0.12 |
| 95 | E. aerogenes GC 7036 TEM-24, Amp C inducable | A (ESBL), C | 2 | 0.06 |
| 96 | E. aerogenes GC 7039 TEM-4, Amp C inducable | A (ESBL), C | >16 | 0.06 |
| 97 | E. cloacae GC 7052 SHV-5, Amp C inducable | A (ESBL), C | 8 | 1 |
| 98 | E. cloacae GC 7065 TEM-26, SHV-12 Amp C inducable | A (ESBL), C | 16 | 0.06 |
| 99 | M. morganii GC 1617 TEM-10 + inducible AmpC | A (ESBL), C | 0.25 | 0.12 |
| 100 | E. coli GC 2149 (ACT-1; TEM-1 + pl 5.6) | A, A (ESBL), C | 16 | 0.50 |
| 101 | E. coli GC 6197 (TEM-1 + SHV-7 + CMY-2) | A, A (ESBL), C | >16 | 4 |
| 102 | K. pneumoniae GC 2825 Act 1 + 3 additional | A, A (ESBL), C | 2 | 0.12 |
| 103 | K. pneumoniae GC 2826 Act 1 + 3 additional | A, A (ESBL), C | 8 | 1 |
| 104 | E. cloacae GC 7055 TEM-1 , SHV-12 Amp C inducable | A, A (ESBL), C | 1 | 0.06 |
| 105 | K. pn GC 2391 (pl 5.4; 5.6; 7.6; 8.7 B-lactamases) | A, A (ESBL), C | 8 | 1 |
| | | (presumed) | | |
| 106 | S. marcescens GC 1781 (Sme-1 + AmpC) | A (Cb'ase), C | 0.06 | 0.06 |
| 107 | E. coli GC 1480 OXA-1 | D | 4 | 0.06 |
| 108 | E. coli GC 1807 OXA-7 | D | 0.12 | 0.03 |
| 109 | E. coli GC 2883 (OXA-10/PSE-2) | D | 0.06 | 0.06 |
| 110 | E. coli GC 4971 OXA-1, TEM-1 | A, D | 2 | 0.12 |
| 111 | E. coli GAR 6649 TEM, OXA, CTX (By PCR only) | A, A (ESBL), D | >16 | 1 |
| 112 | E. coli GAR 5929 TEM, OXA, Act-1 , CTX (By PCR only) | A, C, D | >16 | 0.50 |

*PTZ-R refers to piperacillin-tazobactam-resistant

### Example 3

### In Vivo Antibacterial Protection

Materials:

Animals:

Female mice strain CD-1, approximately 18-22 grams, are received from, e.g., Charles River Laboratories and are quarantined 7 days prior to use. In addition, mice may be rendered neutropenic using cytoxan for particular studies.

Infections:

Clinical isolates that have been adapted to cause infection in mice, are used in the experiment, including infections with strains of E. coli, K. pneumoniae, M. morganli, E. cloacae, S. marcescens, C. freundii, staphylococci, streptococci, P. aeruginosa and N. gonorrhoeae.

Preparation:

Animals are housed five to a cage with free access to food and water, in accordance with NIH guidelines.

Experimental Protocol:

Mice are challenged by injecting 0.5 ml intraperitoneally or 0.05 ml intranasally of a predetermined bacterial inoculum suspended in broth, saline or hog gastric mucin (supplemented with dried bovine hemoglobin for N. gonorrhoeae). The bacterial inoculum is equivalent to 10-100 LD₅₀s of the specific infecting strain and will result in death of the non-treated control animals within 7 days. Antibacterial doses (dose concentration prepared by two fold serial dilutions of the antibiotic) are dissolved or suspended in 0.2% aqueous agar or methocel, phosphate buffered saline or an adjuvant are administered orally, subcutaneously or intravenously in the following manner:

a) Orally or subcutaneously: Dose volume of 0.5 ml administered ½ hr after infection. A second dose may be administered 3 hr. after infection for treatment of infections with more virulent organisms.

b) Intravenously: Dose volume of 0.2 ml, administered ½ hr. after infection. For the treatment of infections with more virulent organisms, more doses, up to 48 hr may be administered. (Intravenous dosing will not exceed 3 doses/24 hr period.)

c) Oral pretreatment: Under special circumstances, the pH of the stomach needs to be adjusted in order to increase the gastric stability of the antibiotic. For this purpose, 0.5 ml of phosphate buffered saline (pH 7.8, 0.06M) (or specific approved adjuvant) is administered orally ½ hr after infection, followed 5 minutes later by 0.5 ml of antibiotic (also orally) contained in phosphate buffered saline (pH 7.8, 0.06M).

Animal Species

A detailed explanation as to the number of animals needed for the determination of in vivo efficacy follows:
A) Novel antibiotics are tested at 5 different dose levels with 5 mice per dose level at each of three routes of administration (oral, subcutaneous and intravenous). Initially the three routes of administration should be investigated so as to determine if the drug is orally absorbed and/or which is the most effective route. This would require 25 mice/route with 3 routes/antibiotic or 75 mice per novel compound tested. One to two novel antibiotics will be tested per experiment (75-150 mice).
B) The effectiveness of the new compound must be compared to that of a standard, or antibiotic of known effectiveness. Known or previously tested antibiotics are tested at 5 dose levels with 5 mice per dose level by a single route of administration, for a total of 25 mice/antibiotic. Usually 3-6 antibiotics will be tested per experiment (75-150 mice).
C) Untreated controls—In each of the above tests, untreated animals are infected with 3 different concentrations of bacterial inoculum with 10 mice per concentration (30 mice total in each and every test). These untreated controls are used to determine and maintain the infection level between 10-100 LD50s as required for test-to-test comparison and validity.

### Example 4

### In Vivo Antibacterial Protection

Materials:

Animals:

Female mice strain CD-1, approximately 18-22 grams, were received from Charles River Laboratories and quarantined 7 days prior to use.

Infections:

Clinical isolates that were adapted to cause infection in mice were used in the experiment, including infections with strains of E. coli and E. cloacae.

Preparation:

Animals were housed five to a cage with free access to food and water, in accordance with NIH guidelines.

Experimental Protocol:

Mice were challenged by injecting 0.5 ml intraperitoneally of a predetermined bacterial inoculum suspended in 5% hog gastric mucin. The bacterial inoculum was equivalent to 10-100 LD₅₀s of the specific infecting strain and was designed to result in death of the non-treated control animals within 7 days. Antibacterial doses (dose concentration prepared by two fold serial dilutions of the antibiotic or antibiotic/compound combination) were dissolved or suspended in 0.2% aqueous agar (subcutaneous administration) or phosphate buffered saline (intravenous administration) and administered intravenously or subcutaneously in the following manner:

a) Subcutaneously: Dose volume of 0.5 ml was administered ½ hr after infection. In the E. cloacae model, a second dose was administered 2.5 hr. after infection, due to the increased virulence of the organism.

b) Intravenously: Dose volume of 0.2 ml, administered ½ hr. after infection. In the E. cloacae model, a second dose was administered 2.5 hr. after infection, due to the increased virulence of the organism.

Animal Species

A detailed explanation of the protocol for the determination of in vivo efficacy follows:
A) The effectiveness of the lacamase inhibitor compound/cefepime combination was evaluated against a cefepime control. The cefepime control was tested at 5 dose levels (0.12, 0.06, 0.03, 0.015, and 0.008 mg/kg in the E. coli model and 2, 1, 0.5, 0.25, and 0.12 mg/kg in the E. cloacae model) with 5 mice per dose level by a single route of administration.

A) Cefepime/lacamase combinations were tested at 5 different dose levels (4:1 cefepime/inhibitor ratio (w/w), based on the cefepime control doses above) with 5 mice per dose level at each of three routes of administration (subcutaneous and intravenous).

C) Untreated controls—In each of the above tests, untreated animals were infected with 3 different concentrations of bacterial inoculum with 10 mice per concentration. Untreated controls were initially tested at the innoculum level, which was sufficient to result in the death of the untreated controls within 48 hours. Additional untreated controls were tested at 1:10 and 1:100 dilutions to determine the LD₅₀. These untreated controls were used to determine and maintain the infection level between 10-100 LD₅₀s as required for test-to-test comparison and validity.

Determination of Protective Effects of Antibacterial Agents:

The protective effects of the antibacterial agent(s) were measured by the survival of the infected untreated as compared to the treated animals. For this determination, animals were observed for 7 days after treatment. A census of survivors was taken twice daily and at that time dead as well as moribund animals are removed. The 7 day survival ratio from three separate tests were pooled for estimation of median effective dose (ED50) by computerized program for probit analysis (Cleeland, R. and E. Squires. 1991. Evaluation of New Antimicrobials in Vitro and in Experimental Animal Infections. In Antibiotics in Laboratory Medicine, 3rd. ed., edited by Victor Lorian. Williams and Wilkins Baltimore, Maryland. pp. 752-783). The test was performed three times on separate days to provide a statistically valid number of animals and to minimize variation in test results on a day-to-day and test-to-test basis.

Results for a combination of cefepime and Compound 1 against E. coli and E. cloacae are shown in Table 3.

TABLE 3

| | | MIC* | ED₅₀ | ED₅₀ |
|---|---|---|---|---|
| Organism | Drug | (μg/mL) | mg/kg/SIV | mg/kg/SSC |
| E. coli GC | Cefipime | 0.06 | 0.039 | |
| 6265 TEM-1 | | | (0.031-0.05) | |
| (LSU-80-8) | | | | |
| | Ceipime + | 0.06 | 0.027 | |
| | Compound 1 | | (0.02-0.035) | |
| | (4:1) | | | |
| E. cloacae | Cefipime | 16 | 4 | 0.74 |
| GC4142 | | | (3.1-5.0) | (0.57-0.98) |
| AmpC | | | | |
| | Ceipime + | 0.12 | 3.3 | 0.63 |
| | Compound 1 | | (2.5-4.3) | (0.49-0.81) |
| | (4:1) | | | |

*minimum inhibitory concentration (lowest dose of antibiotic that inihibits growth of the test organism)

### Example 5

### Synthesis of (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid (Compound 1)

### Preparation of ethyl 6,7-dihydro-5H-cyclopenta[d]imidazo[2,1-b][1,3]thiazole-2-carboxylate

A mixture of 2-chlorocyclopentanone (11.8 g, 100 mmol) and thiourea (8.0 g 101 mmol) was refluxed in ethanol:THF (1:2) for 16 hrs. The reaction mixture was cooled to room temperature and the separated white solid was filtered (9.0 g separated). The white solid was dissolved in anhydrous ethanol (100 ml) and sodium methoxide (2.7 g, 51 mmol) was added. After 15 minutes, ethyl bromopyruvate (10.0 g) was added to the solution, stirred at room temperature for 2 hrs, and then refluxed for 48 hrs. The reaction mixture was cooled to room temperature and concentrated. The residue was extracted with chloroform and washed well with water, and concentrated. The product was purified by silica-gel column chromatography by eluting it with 50% ethyl acetate:hexane. Red semi-solid; Yield: 3.0 g; M+H 237.

The ester was reduced with LiAlH₄ and the resultant alcohol was oxidized with active MnO₂. The aldehyde obtained was taken to the next step.

### Preparation of 4-nitrobenzyl (5R)-6-[(acetyloxy)(6,7-dihydro-5H-cyclopenta[d]imidazo[2,1-b][1,3]thiazol-2-yl)]-6-bromo-7-oxo-4-thia-1-azabicyclo[3.2.0]hept-2-ene-2-carboxylate

2-Formyl-6,7-dihydro-5H-cyclopenta[d]imidazo[2,1-b][1,3]thiazole (600 mg, 3.1 mmol) and the dry THF solution (20 mL) of (5R,6S)-6-bromo-7-oxo-4-thia-1-aza-bicyclo[3.2.0]hept-2-ene-2-carboxylic acid 4-nitro-benzyl ester (1.2 g, 3 mmol) were added successively to the dry acetonitrile (15 mL) solution of anhydrous MgBr₂: O(Et)₂ (1.2 g, 3.0 mmol)under an argon atmosphere at room temperature. After cooling to −20° C., Et₃N (2.0 mL) was added in one portion. The reaction vessel was covered with foil to exclude light. The reaction mixture was stirred for 6 h at −20° C. and treated with acetic anhydride (1.04 mL) in one portion. The reaction mixture was warmed to 0° C. and stirred for 15 h at 0° C. The mixture was diluted with ethyl acetate and washed with 5% citric acid aqueous solution, saturated sodium hydrogen carbonate, and brine. The organic layer was dried (MgSO₄) and filtered through a pad of Celite, washed with ethyl acetate, and the filtrate was concentrated under reduced pressure. The residue was applied to silica gel column chromatography, then the column was eluted with ethyl acetate:hexane (1:1). Collected fractions were concentrated under reduced pressure and the mixture of diastereomers were taken to the next step. Pale yellow amorphous solid; Yield: 850 mg, 45%; M+H 620.

### Preparation of (5R),(6Z)-6-(6,7-dihydro-5H-cyclopenta[d]imidazo[2,1-b][1,3]thiazol-2-ylmethylene)-7-oxo4-thia-1-azabicyclo[3.2.0]hept-2-ene-2-carboxylic acid sodium salt

4-nitrobenzyl (5R)-6-[(acetyloxy)(6,7-dihydro-5H-cyclopenta[d]imidazo[2,1-b][1,3]thiazol-2-yl)]-6-bromo-7-oxo-4-thia-1-azabicyclo[3.2.0]hept-2-ene-2-carboxylate (850 mg, 1.37 mmol) was dissolved in THF (20 mL) and acetonitrile (10 mL). Freshly activated Zn dust (5.2 g) was added rapidly with 0.5 M phosphate buffer (pH 6.5, 28 mL). The reaction vessel was covered with foil to exclude light. The reaction mixture was vigorously stirred for 2 h at room temperature. The reaction mixture was filtered, cooled to 3° C., and 0.1 N NaOH was added to adjust the pH to 8.5. The filtrate was washed with ethyl acetate and the aqueous layer was separated. The aqueous layer was concentrated under high vacuum at 35° C. to give a yellow precipitate. The product was purified by HP21 resin reverse phase column chromatography. Initially the column was eluted with deionized water (2 L) and latter with 10% acetonitrile:water. The fractions containing the product were collected and concentrated under reduced pressure at room temperature. The yellow solid was washed with acetone, filtered and dried. Yield: 138 mg, 29%; as yellow crystals; mp 192° C.; (M+H+Na) 367. ¹H NMR (DMSO-d₆) δ 2.51 (m, 4H), 3.01 (m, 2H), 8.2 (s, 1H), 7.1 (s, 1H), 6.55 (s, 1H), 6.4 (s, 1H).

Alternatively 4-nitrobenzyl (5R)-6-[(acetyloxy)(6,7-dihydro-5H-cyclopenta[d]imidazo[2,1-b][1,3]thiazol-2-yl)]-6-bromo-7-oxo-4-thia-1-azabicyclo[3.2.0]hept-2-ene-2-carboxylate was hydrogenated at 40 psi for three hours in acetonitrile and 0.5 M phosphate buffer (pH 6.5, 10 mL). The reaction mixture was filtered, cooled to 3° C., and 0.1 N NaOH was added to adjust the pH to 8.5. The filtrate was washed with ethyl acetate and the aqueous layer was separated. The aqueous layer was concentrated under high vacuum at 35° C. to give a yellow precipitate. The product was purified by HP21 resin reverse phase column chromatography. Initially the column was eluted with deionized water (2 L) and latter with 10% acetonitrile:water. The fractions containing the product were collected and concentrated under reduced pressure at room temperature. The yellow solid was washed with acetone, filtered and dried. Obtained the desired material (yellow solid) in 24% yield. Mp: 189° C. ¹H NMR (DMSO) δ 2.51 (m, 4H), 3.01 (m, 2H), 8.2 (s, 1H), 7.1 (s, 1H), 6.55 (s, 1H), 6.4 (s, 1H).

### Example 6

### Synthesis of (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid (Compound 2)

### Preparation of ethyl 5,8-dihydro-6H-imidazo[2,1-b]pyrano[4,3-d][1,3]thiazole-2-carboxylate

SO₂Cl₂ (7.4 g, 55 mmol) was slowly added to a mixture of tetrahydro-4H-pyran-4-one (5.0 g, 50 mmol) in CCl₄ (100 ml) at 0° C. After the addition, the reaction mixture was stirred at room temperature for 4 hrs and carefully quenched with ice-cold water. The reaction mixture was washed well and dried over anhydrous MgSO₄. The organic layer was filtered and concentrated. The colorless oil obtained was dissolved in THF/EtOH containing thiourea (4.0 g, 52 mmol) and refluxed for 8 hrs. The reaction mixture was then cooled to room temperature and the separated 6,7-dihydro-4H-pyrano[4,3-d][1,3]thiazol-2-amine hydrochloride white solid was filtered. Yield. 4.5 g (47%); M.Pt. 115° C., (M+H) 157.

6,7-dihydro-4H-pyrano[4,3-d][1,3]thiazol-2-amine hydrochloride (4.0 g, 20.8 mmol) was dissolved in anhydrous ethanol (100 ml) and sodium methoxide (1.1 g, 21 mmol) was added. The reaction mixture was stirred at room temperature for 30 minutes, then ethyl bromopyruvate (10.0 g) was added and stirred at room temperature for 2 hrs. The reaction mixture was refluxed for 48 hrs, and subsequently cooled to room temperature and concentrated. The residue was extracted with chloroform and washed well with water. The product was purified by silica-gel column chromatography by eluting it with 50% ethyl acetate: hexane. Red semi-solid; Yield: 3.1 g, (59%) M+H 253.

The ester was reduced with LiBH₄ and the resultant alcohol was oxidized with active MnO₂. The aldehyde obtained was taken to next step.

### Preparation of 4-nitrobenzyl (5R)-6-[(acetyloxy)(5,8-dihydro-6H-imidazo[2,1-b][1,3]pyrano[4,3-d][1,3]thiazol-2-yl)]-6-bromo-7-oxo-4-thia-1-azabicyclo[3.2.0]hept-2-ene-2-carboxylate

2-Formyl-5,8-dihydro-6H-imidazo[2.1-b]pyrano[4,3-d][1,3]thiazole (208 mg, 1.0 mmol) and a dry THF solution (20 mL) of (5R,6S)-6-bromo-7-oxo-4-thia-1-aza-bicyclo[3.2.0]hept-2-ene-2-carboxylic acid 4-nitro-benzyl ester (400 mg, 1.1 mmol) were added successively to a dry acetonitrile (15 mL) solution of anhydrous MgBr₂:O(Et)₂ (1.2 g, 3.0 mmol) under an argon atmosphere at room temperature. After cooling to −20° C., Et₃N (2.0 mL) was added in one portion. The reaction vessel was covered with foil to exclude light. The reaction mixture was stirred for 6 h at −20° C. and treated with acetic anhydride (1.04 mL) in one portion. The reaction mixture was warmed to 0° C. and stirred for 15 h at 0° C. The mixture was diluted with ethyl acetate and washed with 5% citric acid aqueous solution, saturated sodium hydrogen carbonate, and brine. The organic layer was dried (MgSO₄) and filtered through a pad of Celite. The pad was washed with ethyl acetate. The filtrate was concentrated under reduced pressure. The residue was applied to a silica gel column, and the column was eluted with ethyl acetate:hexane (1:1). Collected fractions were concentrated under reduced pressure and the mixture of diastereomers were taken to the next step. Pale yellow amorphous solid; Yield: 400 mg, 62%; M.Pt. 78° C.; M+H 636.

### Preparation of (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt

4-nitrobenzyl (5R)-6-[(acetyloxy)(5,8-dihydro-6H-imidazo[2,1-b][1,3]pyrano[4,3-d][1,3]thiazol-2-yl)]-6-bromo-7-oxo-4-thia-1-azabicyclo[3.2.0]hept-2-ene-2-carboxylate (500 mg, 0.79 mmol) was dissolved in THF (20 mL) and acetonitrile (10 mL). Freshly activated Zn dust (5.2 g) was added rapidly with 0.5 M phosphate buffer (pH 6.5, 28 mL). The reaction vessel was covered with foil to exclude light. The reaction mixture was vigorously stirred for 2 h at room temperature. The reaction mixture was filtered, cooled to 3° C., and 0.1 N NaOH was added to adjust the pH to 8.5. The filtrate was washed with ethyl acetate and the aqueous layer was separated. The aqueous layer was concentrated under high vacuum at 35° C. to give a yellow precipitate. The product was purified by HP21 resin reverse phase column chromatography. Initially the column was eluted with deionized water (2 L) and latter with 10% acetonitrile:water. The fractions containing the product were collected and concentrated under reduced pressure at room temperature. The yellow solid was washed with acetone, filtered and dried. Yield: 85 mg, 30%; as yellow crystals; mp 205° C.; (M+H+Na) 383 .¹H NMR (DMSO-d₆) δ 2.8 (m, 2H), 4.0 (m,2H), 4.6(s,2H), 6.4 (s, 1H), 6.5 (s, 1H), 7.0 (s, 1H), 8.1 (s, 1H).

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A method for treating a bacterial infection or disease comprising providing to a patient in need thereof an effective amount of cefepime or a pharmaceutically acceptable salt thereof and a compound of formula I: 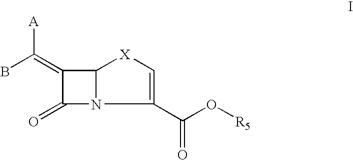
or a pharmaceutically acceptable salt or in vivo hydrolysable ester thereof wherein:
one of A and B denotes hydrogen and the other of A and B denotes an optionally substituted fused tricyclic heteroaryl group;
X is S or O;
R₅ is hydrogen, C₁-C₆ alkyl, C₅-C₆ cycloalkyl, or CHR₃OCOC₁-C₆alkyl; and
R₃ is hydrogen, C₁-C₆ alkyl, C₅-C₆ cycloalkyl, optionally substituted aryl, or optionally substituted heteroaryl.

2. The method of claim 1, wherein the tricyclic heteroaryl group has the formula 16-A: 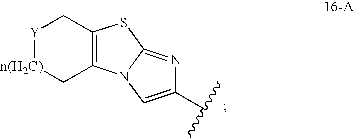
wherein Y is O or CH₂; and n is 0 or 1.

3. The method of claim 2, wherein Y is O and n is 1.

4. The method of claim 2, wherein Y is CH₂ and n is 0.

5. The method of claim 1, wherein the compound of formula I is (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid sodium salt; or (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt.

6. The method of claim 1, comprising co-administering cefepime or a pharmaceutically acceptable salt thereof and the compound of formula I or pharmaceutically acceptable salt or in vivo hydrolysable ester thereof.

7. The method of claim 1, wherein the ratio of cefepime or a pharmaceutically acceptable salt thereof to the compound of formula I or pharmaceutically acceptable salt or in vivo hydrolysable ester thereof is from about 1:1 to about 100:1.

8. The method of claim 1, wherein the ratio of the cefepime or a pharmaceutically acceptable salt thereof to the compound of formula I or pharmaceutically acceptable salt or in vivo hydrolysable ester thereof is less than about 10:1.

9. The method of claim 1, comprising orally administering to a patient.

10. The method of claim 1, comprising intravenously administering to a patient.

11. A composition comprising a pharmaceutically acceptable carrier, cefepime or a pharmaceutically acceptable salt thereof, and a compound of formula I: 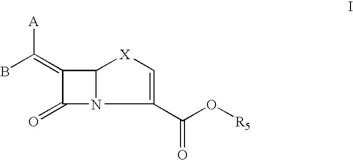
or a pharmaceutically acceptable salt or in vivo hydrolysable ester thereof wherein:
one of A and B denotes hydrogen and the other of A and B denotes an optionally substituted fused tricyclic heteroaryl group;
X is S or O;
R₅ is hydrogen, C₁-C₆ alkyl, C₅-C₆ cycloalkyl, or CHR₃OCOC₁-C₆alkyl; and
R₃ is hydrogen, C₁-C₆ alkyl, C₅-C₆ cycloalkyl, optionally substituted aryl, or optionally substituted heteroaryl.

12. The composition of claim 11, wherein the tricyclic heteroaryl group has the formula 16-A: 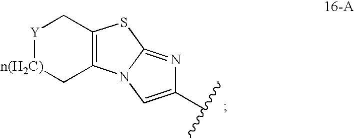
or a pharmaceutically acceptable salt or in vivo hydrolysable ester thereof, wherein Y is O or CH₂; and n is 0 or 1.

13. The composition of claim 12, wherein Y is O and n is 1.

14. The composition of claim 12, wherein Y is CH₂ and n is 0.

15. The composition of claim 11, wherein the compound of formula I is (5R),(6Z)-6-(6,7-dihydro-5H-cyclopenta[d]imidazo[2,1-b][1,3]thiazol-2-ylmethylene)-7-oxo-4-thia-1-azabicyclo[3.2.0]hept-2-ene-2-carboxylic acid sodium salt; or (5R),(6Z)-6-(5,8-dihydro-6H-imidazo[2,1-b]pyrano[4,3-d][1,3]thiazol-2-ylmethylene)-7-oxo-4-thia-1-azabicyclo[3.2.0]hept-2-ene-2-carboxylic acid sodium salt.

16. The composition of claim 11, wherein the ratio of cefepime or a pharmaceutically acceptable salt thereof to the compound of formula I or pharmaceutically acceptable salt or in vivo hydrolysable ester thereof is from about 1:1 to about 100:1.

17. The composition of claim 11, wherein the ratio of the cefepime or a pharmaceutically acceptable salt thereof to the compound of formula I or pharmaceutically acceptable salt or in vivo hydrolysable ester thereof is less than about 10:1.

18. A package comprising a pharmaceutically acceptable carrier, cefepime or a pharmaceutically acceptable salt thereof, a compound of formula I: 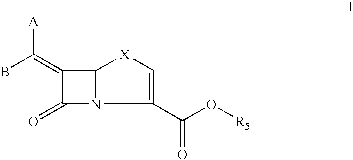
or a pharmaceutically acceptable salt or in vivo hydrolysable ester thereof wherein:
one of A and B denotes hydrogen and the other of A and B denotes an optionally substituted fused tricyclic heteroaryl group;
X is S or O;
R₅ is hydrogen, C₁-C₆ alkyl, C₅-C₆ cycloalkyl, or CHR₃OCOC₁-C₆alkyl; and
R₃ is hydrogen, C₁-C₆ alkyl, C₅-C₆ cycloalkyl, optionally substituted aryl, or optionally substituted heteroaryl
and instructions, wherein the instructions comprise instructions for treating a bacterial infection or disease.
